SEQUENCE LISTING (22 sequences)

SEQ ID NO: 1 - protein, 12 aa, Artificial Sequence
  Synthetic or natural peptide, template for peptides used in the sequence listing
KKRYSREFLLGF

SEQ ID NO: 2 - protein, 12 aa, Artificial Sequence
  Synthetic sequence
KKRYSRXFLLXF

SEQ ID NO: 3 - protein, 12 aa, Artificial Sequence
  Synthetic sequence
KKRYSREXLLGX

SEQ ID NO: 4 - protein, 12 aa, Artificial sequence
  Synthetic Sequence
KKRYSREXLLXF

SEQ ID NO: 5 - protein, 12 aa, Artificial sequence
  Synthetic sequence
KKRYSRXQLLXL

SEQ ID NO: 6 - protein, 11 aa, Artificial sequence
  Synthetic sequence
KKRYSRXFLLX

SEQ ID NO: 7 - protein, 12 aa, Artificial Sequence
  Synthetic sequence
KKRYSRXFLLXX

SEQ ID NO: 8 - protein, 12 aa, Artificial sequence
  Synthetic sequence
KKRYSRXALLXF

SEQ ID NO: 9 - protein, 12 aa, Artificial sequence
  Synthetic sequence
KKRYSRXFLLXF

SEQ ID NO: 10 - protein, 12 aa, Artificial Sequence
  Synthetic sequence
KKRYSREXLLGX

SEQ ID NO: 11 - protein, 12 aa, Artificial sequence
  Synthetic sequence
KKRYSREXLLXF

SEQ ID NO: 12 - protein, 12 aa, Artificial sequence
  Synthetic sequence
KKRYSRXQLLXL

SEQ ID NO: 13 - protein, 11 aa, Artificial Sequence
  Synthetic sequence
KKRYSRXFLLX

SEQ ID NO: 14 - protein, 12 aa, Artificial Sequence
  Synthetic sequence
KKRYSRXFLLXX

SEQ ID NO: 15 - protein, 12 aa, Artificial Sequence
  Synthetic sequence
KKRYSRXALLXF

SEQ ID NO: 16 - protein, 14 aa, Artificial Sequence
  Synthetic sequence
KKRYSREFLLGFQF

SEQ ID NO: 17 - protein, 14 aa, Artificial Sequence
  Synthetic sequence
KKRYSRXFLLGFQX

SEQ ID NO: 18 - protein, 14 aa, Artificial Sequence
  Synthetic sequence
KKRYSXEFLLGFXF

SEQ ID NO: 19 - protein, 14 aa, Artificial Sequence
  Synthetic sequence
KKRYXREFLLGXQF

SEQ ID NO: 20 - protein, 15 aa, Artificial Sequence
  Synthetic sequence
KKRYSREXLLGFQFX

SEQ ID NO: 21 - protein, 15 aa, Artificial Sequence
  Synthetic sequence
KKRYXXXXLLXXXXX

SEQ ID NO: 22 - protein, 12 aa, Artificial Sequence
  Synthetic sequence
KKRYSRXXLLXX

(19) United States
(12) Patent Application Publication
(10) Pub. No.: US 2016/0046672 A1
(43) Pub. Date: Feb. 18, 2016

(54) STAPLING eIF4E INTERACTING PEPTIDES

(71) Applicant: AGENCY FOR SCIENCE, TECHNOLOGY AND RESEARCH, Singapore (SG)

(72) Inventors: Christopher John Brown, Singapore (SG); David Philip Lane, Singapore (SG); Soo Tng Quah, Singapore (SG); Dilraj Lama, Singapore (SG); Chandra Shekhar Verma, Singapore (SG)

(21) Appl. No.: 14/778,761

(22) PCT Filed: Feb. 28, 2014
(86) PCT No.: PCT/SG2014/000094

§ 371 Date: Sep. 21, 2015

(30) Foreign Application Priority Data
Mar. 21, 2013 (SG) 201302065-6
Jan. 15, 2014 (SG) 201400336-2

(51) Int. Cl. C07K 7/08

(52) U.S. Cl. CPC C07K 7/08

(57) ABSTRACT

The present invention relates to cross-linked peptides that are associated with human eIF4G and bind to eIF4E, uses thereof and pharmaceutical compositions comprising the peptides.

### CROSS-REFERENCE TO RELATED APPLICATIONS

This application claims the benefit of priority of Singapore application No. 201302065-6 filed Mar. 21, 2013 and Singapore application no. 201400336-2 filed on Jan. 15, 2014, the contents of it being hereby incorporated by reference in its entirety for all purposes.

### FIELD OF THE INVENTION

The present invention lies in the field of molecular biology and relates to cross-linked peptides and pharmaceutical uses thereof.

### BACKGROUND OF THE INVENTION

The human eukaryotic translation initiation factor 4E (eIF4E) initiates cap-dependent translation by binding to the cap structure (m⁷GTP) found at the 5′ end of mRNA. eIF4F is frequently over-expressed in a large number of cancers and results in the increased translation of oncogenic proteins via deregulated cap-dependent translation. Inhibitors of the eIF4E:eIF4G interactions represents a viable approach that would normalize cap-dependent translation in cancer cells.

mRNAs are hypothesized to compete with one another for binding to the eIF4F (eukaryotic translation initiation factor 4F) protein complex for delivery to the ribosomes and subsequent translation eIF4F forms a complex with the 40S ribosomal subunit and eIF3. This complex shuttles along the 5′-untranslated region (5′-UTR) of the mRNA until it arrives at the AUG initiation codon. The short, unstructured 5′-UTRs of most cellular mRNAs enable the eIF4E containing complex to scan efficiently for the translation initiation codon (AUG). In comparison, the lengthy, G+C-rich, highly structured 5′-UTRs typical of proto-oncogenic mRNAs (e.g. cyclin D1, VEGF) hinder recognition of the AUG start codon by the initiation complex and leads to mRNAs being translated poorly. eIF4E contributes to malignancy by enabling the increased translation of mRNAs with highly structured 5′UTRs either when over-expressed or when the eIF4F complex is not regulated correctly.

A recent report has indicated that the small molecule ribavirin might interfere with the eIF4E:cap interaction and may therefore present a clinical opportunity as an eIF4E-targeted therapy. As anticipated, ribavirin treatment selectively diminished the expression of key, eIF4E-dependent proteins such as cyclin D1 and suppressed tumor growth. However, whether or not ribavirin actually binds eIF4E is controversial. Consequently, a more directed approach to develop small molecule inhibitors of the eIF4E: 7-methylguanosine cap interaction might be a fruitful approach for the development of an eIF4E-specific small molecule therapy. To date, no such drug-like inhibitors of the eIF4E-cap interaction have been reported.

Thus, there is a need to provide new peptides with affinity for eIF4E that overcome, or at least ameliorate, one or more of the disadvantages described above.

### SUMMARY OF THE INVENTION

Described below are cross-linked peptides related to a portion of human eIF4E. These cross-linked peptides contain at least two modified amino acids that together form an internal cross-link (also referred to as a staple) that can help to stabilize the alpha-helical secondary structure of a portion of eIF4G1 that is thought to be important for binding of eIF4E to eIF4G. Accordingly, a cross-linked peptide described herein can have improved biological activity relative to a corresponding peptide that is not cross-linked. The cross-linked eIF4G1 peptides are thought to interfere with binding of eIF4G1 to eIF4G thereby inhibiting the increased translation of mRNAs with highly structured 5′UTRs. The cross-linked eIF4G1 peptide described herein can be used therapeutically, e.g., to treat or prevent a variety of cancers in a subject. For example, cancers or other disorders characterized by an undesirably high level or high activity of eIF4E and/or cancers or other disorders characterized by an undesirably high level of activity of eIF4E containing complexes.

Thus, in a first aspect, there is provided an isolated peptide comprising or consisting of the amino acid sequence of:

(SEQ ID NO: 21)
K¹K²R³Y⁴Xaa₁Xaa₂Xaa₃Xaa₄L⁹L¹⁰Xaa₅Xaa₆Xaa₇Xaa₈Xaa₉

wherein:
Xaa₁ is selected from the group consisting of S (serine), aminoisobutyric acid and an unnatural amino acid;
Xaa₂ is selected from the group consisting of R (arginine), aminoisobutyric acid and an unnatural amino acid;
Xaa₃ is selected from the group consisting of E (glutamic acid), aminoisobutyric acid and an unnatural amino acid;
Xaa₄ is selected from the group consisting of F (phenylalanine), Q (glutamine), A (alanine), aminoisobutyric acid and an unnatural amino acid;
Xaa₅ is selected from the group consisting of G, aminoisobutyric acid and an unnatural amino acid;
Xaa₆ is selected from the group consisting of F (phenylalanine), L (leucine), aminoisobutyric acid, 2-aminobutyric acid and an unnatural amino acid;
Xaa₇ is absent or selected from the group consisting of Q (glutamine), aminoisobutyric acid and an unnatural amino acid;
Xaa₈ is absent or selected from the group consisting of F (phenylalanine), aminoisobutyric acid and an unnatural amino acid;
Xaa₉ is absent or selected from the group consisting of aminoisobutyric acid and an unnatural amino acid;
wherein the peptide comprises at least one peptide-cross linker linking Xaa₁, Xaa₂, Xaa₃ or Xaa₄ with Xaa₅, Xaa₆, Xaa₇, Xaa₈ or Xaa₉.

In a second aspect, there is provided an isolated peptide comprising the amino acid sequence of:

(SEQ ID NO: 22)
K¹K²R³Y⁴S⁵R⁶Xaa₁Xaa₂L⁹L¹⁰Xaa₃Xaa₄

wherein:
Xaa₁ is selected from the group consisting of E (glutamic acid), aminoisobutyric acid and an unnatural amino acid;
Xaa₂ is selected from the group consisting of F (phenylalanine), Q (glutamine), A (alanine), aminoisobutyric acid and an unnatural amino acid;
Xaa₃ is selected from the group consisting of G, aminoisobutyric acid and an unnatural amino acid;
Xaa₄ is selected from the group consisting of F (phenylalanine), L (leucine), aminoisobutyric acid, 2-aminobutyric acid and an unnatural amino acid;
wherein the peptide comprises at least one peptide-cross linker linking Xaa₁ or Xaa₂ with Xaa₃ or Xaa₄.

In a third aspect, there is provided an isolated nucleic acid molecule encoding KKRYSREFLLGF (SEQ ID NO: 1) and modified to obtain any one of the peptides described herein.

In a fourth aspect, there is provided a vector comprising a nucleic acid molecule as described above.

In a fifth aspect, there is provided a host cell comprising a nucleic acid molecule or a vector as described herein.

In a sixth aspect, there is provided a pharmaceutical composition comprising a peptide as described herein, or an isolated nucleic acid molecule as described herein, or a vector as described herein.

In a seventh aspect, there is provided the use of the peptide disclosed herein in the manufacture of a medicament for treating or preventing cancer.

In an eighth aspect, there is provided a method of treating or preventing cancer in a patient comprising administering a pharmaceutically effective amount of the peptide disclosed herein or the isolated nucleic acid molecule disclosed herein, or the vector disclosed herein.

### BRIEF DESCRIPTION OF THE DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 shows representative snapshots from simulations of A) sTIP-01:eIF4E showing the displacement of F8 due to steric occlusion. The sterically occluded F8 side-chain rotates around the χ-2 torsion angle and buries itself, quite favourably, against the surface of eIF4E. The F8 side chain now impedes Y4 from maintaining the conserved hydrogen bond with the backbone carbonyl of P38, causing Y4 to ‘flip out’ and become more exposed to the solvent, thereby reducing its energetic contribution to peptide:protein interactions. B) TIP-01:eIF4E highlighting that no such conformational changes for F8 occur here, explaining the higher energetic contribution to peptide:protein interactions and thus the higher K_{d}. C) sTIP-02:eIF4E showing changes associated with the replacement of F8 and F12 with an i, i+4 staple. It shows that the staple interacts with the protein and contributes favourably to binding. However, the lack in improvement of affinity suggests that the stapled peptide does not optimally replace the influence of F8 and F12. D) TIP-02:eIF4E illustrating packing of Y4 into the space previously occupied by F8. Y4 undergoes a conformational change and packs into the space previously occupied by F8 in the linear peptide and the staple in sTIP-02. This results in the loss of the Y4 hydrogen bond and weakens the interaction energy, although the L9:W73 h-bond remains unaffected by these new interactions

FIG. 2 depicts under A) the crystal structure of the eIF4G1^{D5S} peptide in complex with eIF4E. (PDB ID: 4AZA). The crystal structure of the eIF4G^{D5S} peptide bound to eIF4E was examined to identify sites for the insertion of a staple. The tyrosine (Y4) is engaged in multiple van der Waal contacts with eIF4E and an h-bond between its side chain hydroxyl and the carbonyl backbone of P38 of eIF4E. The leucine (L9) exploits a shallow cavity on the surface of eIF4E and interacts with W73 of eIF4E via an h-bond between its backbone and the indole of the tryptophan. The conserved hydrophobic residue (L10) packs against L131 and L135 of eIF4E. Crystal structures of both peptides complexed to eIF4E are approximately 50% α-helical; however they contain negligible helical content in solution. Protein is shown in surface and the peptide in cartoon representation. All residues from the peptide are shown in stick and labeled. Hydrogen bond between Y4:P38 and L9:W73 are represented.

FIG. 2 under B) is a representative snapshot from the computer simulation of eIF4G1^{D5S} in complex with eIF4E showing the lack of stable hydrogen bond between Y4:P38 as observed in the crystal structure even without the disruption imposed upon this interaction by the conformational changes in sTIP-01.

FIG. 2 under C) shows on the upper panel all 3 linkages in models of the eIF4E interacting sTIP-01, 02 and 03 peptides, respectively. The lower panel shows the structures of the hydrocarbon linkages incorporated into the peptides sequences. Staple shown in orange. Xᵣ=(R)-2-(4′-pentenyl) alanine and Xₛ=and (S)-2-(4′-pentenyl) alanine. eIF4E interacting peptides were stapled via either an I, I+4, I, I+3 or I, I+7 linkage between either positions 7 and 11, 8 and 12, 8 and 11, 7 and 14, 6 and 13, 5 and 12 or 8 and 15.

FIG. 3 depicts representative snapshots from simulations of A) sTIP-03:eIF4E complex showing formation of the Y4:P38 h-bond and packing of H37 with Y4 and F12. The restrained C-terminal F12 predominately packs against H37, which also forms van der Waals contacts with Y4. B) TIP-03eIF4E complex illustrating a binding mode that is similar to that of sTIP-03 with eIF4E. The association of the conformationally more labile, diAIB analogue peptide (TIP-03) with eIF4E is characterized by an interaction network between F12, H37 and Y4 similar to that in sTIP-03.

FIG. 4 shows representative snapshots from simulations of A) sTIP-01^{F8A}:eIF4E depicting the “in” conformational state where it forms a stacking interaction with Y4 and F12 and the absence of the Y4:P38 h-bond. B) TIP-01^{F8A}:eIF4E showing H37 interacting favourably with Y4 and their lack of interactions with F12. Simulations of sTIP-01^{F8A} and TIP-01^{F8A} reveal that the interaction pattern between Y4, H37 and F12 influence the stability of the Y4:P38 h-bond. C) sTIP-01^{F12&}:eIF4E complex revealing that by introducing the staple the interactions of 2AB change, causing the packing of H37 and F8 to alter significantly. D) TIP-01^{F12&}:eIF4E showing H37 interacting favourably with Y4 but with F8 rotating to pack against eIF4E due to the interaction of 2AB. The Y4:P38 h-bond remains highly stable in simulations of the sTIP/TIP-01F^{12&} derivative peptides. In TIP-01F^{12&}, the C-terminal 2AB forms no interactions with H37. Instead H37 forms hydrophobic interactions with Y4 and causes no disruption of the h-bond. The incorporation of the i, i+4 staple induces a conformational change in the interactions formed by the peptide by restraining the C-terminal region of the helix. This causes 2AB to interact predominantly with H37 which in turn stacks with F8 resulting in a similar mode of binding as in eIF4G^{D5S}.

FIG. 5 under A) is a representation of the crystal structure of sTIP-04:eIF4E showing the 2Fo-Fc map for the peptide ligand as a 1.5 cut off. The S5 side-chain forms an interaction network with the Q8 side-chain and the backbone amides on the first turn of the peptide helix, thus stabilizing the bound complex. Simulations showed that the L9:W73 hydrogen bond in both derivative peptides (sTIP-04 and TIP-04) is very stable.

FIG. 5 under B) depicts a representative snapshot of the TIP-04:eIF4E complex illustrating maintenance of the Q8:S5 interaction network, existence of the Y4:P38 h-bond and more optimal packing of L12 with H37. In TIP-04 the optimal packing of H37, L12 and Y4 does not disrupt the conserved hydrogen bond. In the stapled derivative, H37 forms more favourable van der waals contacts with L12, as a result of the staple rigidifying the C-terminal, which causes Y4 to undergo a transition in order to maintain favourable packing. It is this favourable packing rearrangement as can be seen from the energetic contribution of Y4 that causes the attenuation of the Y4:P38 h-bond.

FIG. 6 shows circular dichroism spectra of TIP and sTIP variant peptides. The CD spectra reveal that the staple induces greater helicity in sTIP-01 than in TIP-01 or in eIF4G^{D5S}.

FIG. 7 is a plot showing the Chi2 (χ2) angle of F8 sidechain in the sTIP-01 computer simulation. The covalent staple in sTIP-01 imposes rigidity in the α-helix, increasing the strain on the network of interactions formed between H37, F8 and F12. This leads to steric occlusion of F8, causing a series of conformational changes to propagate along the peptide:protein interface. The sterically occluded F8 side-chain rotates around the χ2 torsion angle and buries itself, quite favourably.

FIG. 8 shows a representative snapshot from the computer simulation of sTIP-01F8A in complex with eIF4E illustrating H37 in the ‘out’ position, Y4 occupying the space vacated due to Y8A mutation and the packing of F12 against. Y4. H37 can be found in the alternative ‘in’ state and the conformational changes result in the rare formation of the h-bond. Hence, H37 and F12 influence the stability of the Y4:P38 h-bond.

FIG. 9 shows representative snapshots from the computer simulations of A) sTIP-01Tr in complex with eIF4E and B) TIP-01Tr in complex with eIF4E showing that when the C-terminal F12 is removed that contrasting rearrangement of the packing interactions of F8, Y4 and H37 result, which are dependent on whether or not a macrocyclic linkage is present.

FIG. 10 shows representative snapshots from simulations of sTIP-04:eIF4E initiated from two different conformations. A) The starting state derived from eIF4E^{D5S} (PDB ID: 4AZA) complex structure and B) simulation started from eIF4E: sTIP-04 crystal structure (PDB ID: 4BEA.PDB). Both simulations are in good overall agreement with each showing the same structural features in terms of the intra/inter-molecular interactions which involve the Q8-S5 interaction network, optimal packing of L12 and the loss of Y4:P38 hydrogen bond.

FIG. 11 is a Table (Table 3) summarizing the total free energy decomposition of peptide residues across simulated systems.

FIG. 12A is a dot plot representing normalized luminescence in MDA-MB-468 and MDA-MB-231 cells over increased concentration of staple peptides. MDA-MB-468 and MDA-MB-231 cells were lysed and a recombinant luciferase protein was added to the lysed cells. Subsequently, the cells were incubated with the indicated concentration of s-TIP03 and a control staple peptide showing that s-TIP03 decreases cell viability in a dose-dependent manner.

FIG. 12B shows representative images of Western Blot representing protein levels of eIF4e, Survivin, Bcl-XL and actin in cell extracts from MDA-MB-231 cells grown in the absence or presence of 10% Fetal calf serum, that were treated with the indicated concentration of stapled peptides previously diluted in 100% Dimethylsulfoxide (DMSO) to achieve a final concentration of DMSO of 1%. sTIP-03 down-regulates survivin and Bcl-XL protein levels in MDA-MB-231 cells in a dose-dependent manner. Actin protein is a loading control to indicate that the same amount of proteins was loaded into each well.

### DETAILED DESCRIPTION OF THE PRESENT INVENTION

Before the present peptides and uses thereof are described, it is to be understood that this invention is not limited to particular peptides, methods, uses and experimental conditions described, as such peptides, methods, uses and conditions may vary. It is also to be understood that the terminology used herein is for purposes of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

In the present invention, isolated cross-linked peptides have been designed rationally to interrupt the eIF4E-eIF4G interface. Biophysical data and crystal structure were used to support molecular dynamic simulations of a set of isolated peptides. The inventors found that the peptides described herein bind with an apparent K_{d} of single digit nanomolar range, corresponding to a ˜17 to ˜25-fold improvement of the K_{d} over the linear template that was used to design the peptides of the invention. In addition, the inventors found the structural effects that can occur at peptide:protein interfaces, which mutually modulate each other when conformational freedom is reduced by the introduction of a covalent staple linkage in the peptides. In other words, the inventors found that alternative helical stabilisation strategies give rise to diverse molecular mechanisms for binding and that improvements in affinity result from compensatory interactions.

Peptides cross-linkers predominately increase the helicity of the peptide in solution before binding but this can be compromised by non-optimal interactions at the peptide:protein interface. In the rationally designed peptides of the invention such limitations have been overcome, or at least ameliorated by optimising packing effects at the interface, stabilising the bound complex and greater helical stabilization in solution. For example in exemplary peptide of the invention, the cross-linker only induces 45% helicity but this is compensated for with the formation of the (hydrogen) h-bond between two amino acids and by optimal packing interactions of another amino acid of the peptide. In contrast, another exemplary peptide may lose the hydrogen bond between the two amino acids upon binding but compensation arises via greater helicity (63%) in solution and stabilisation of the helical bound form by another amino acid. This is reflected in the enthalpy and entropy values of binding derived for these two peptides with the first exemplary peptide having a more favourable enthalpic component and the second exemplary peptide having a more favourable entropic component.

The inventors found that an isolated peptide of the present invention is a potent binder of eIF4E compared to other inhibitors known to the skilled artisan. The observations made by the inventors and disclosed herein are useful in the design of new eIF4E inhibitors for therapeutic applications, for example, in the treatment of cancer.

An alternative approach to targeting the eIF4E-cap interaction is to selectively disrupt the interaction of eIF4E with eIF4G, thereby disabling the formation of the eIF4F complex. An alternative approach to targeting eIF4E would be to reduce eIF4E protein expression using antisense oligonucleotides (ASOs). eIF4E ASOs have been shown to effectively reduce both eIF4E RNA and protein in a wide array of transfected human and murine cells, subsequently reducing the expression of the malignancy-related proteins-specifically cyclin D1, VEGF, c-myc, survivin and BCL-2. Importantly, ASO mediated reduction of eIF4E did not affect the expression of β-actin, a protein encoded by a “strong” mRNA nor did it reduce overall, protein synthesis substantially.

Peptidomimetics represent an alternative approach to targeting eIF4E:eIF4G interaction. Proteins in their natural state are folded into regions of secondary structure, such as helices, sheets and turns. The alpha-helix is one of the most common structural motifs found in the proteins, and many biologically important protein interactions are mediated by the interaction of an α-helical region of one protein with another protein. Yet, α-helices have a propensity for unraveling and forming random coils, which are, in most cases, biologically less active, or even inactive, have lower affinity for their target, have decreased cellular uptake and are highly susceptible to proteolytic degradation.

Thus, the present invention relates to an isolated peptide that may comprise or consist of the amino acid sequence set forth in SEQ ID NO: 21 (K¹K²R³Y⁴Xaa₁Xaa₂Xaa₃Xaa₄L⁹L¹⁰Xaa₅Xaa₆Xaa₇Xaa₈Xaa₉). The peptides may include at least one peptide cross-linker (also called a staple or a tether) between two non-natural (i.e. unnatural or synthetic) amino acids that significantly enhance the alpha helical structure of the peptides. Generally, the cross-linker extends across the length of one or two helical turns (that is about 3.4 or about 7 amino acids). Accordingly, amino acids positioned at i and i+3 (3 amino acids apart); and i and i+4; or i and i+7 are ideal candidates for chemical modification and cross-linking.

Thus for example, where a peptide has the sequence: [ . . . ]XaaᵢXaaⱼXaaₖXaaₗXaaₘXaaₙXaaₒXaaₚXaa_{q}Xaaᵣ[ . . . ] (wherein “[ . . . ]” denotes the optional presence of additional amino acids), cross-linkers between Xaaᵢ and Xaaₗ, or between Xaaᵢ and Xaaₘ, or between Xaaᵢ and Xaaₚ are useful as are cross-linkers between Xaaᵢ and Xaaₘ, or between Xaaⱼ and Xaaₙ, or between Xaaⱼ and Xaa_{q}, etc. . . . . The peptides may include more than one cross-linker to either further stabilize the sequence or facilitate the stabilization of longer peptide stretches.

Thus, in one aspect the present invention refers to the isolated peptide described above wherein the peptide comprises at least one cross-linker Xaa₁, Xaa₂, Xaa₃ or Xaa₄ with Xaa₅, Xaa₆, Xaa₇, Xaa₈ or Xaa₉, and wherein Xaa₁ includes, but is not limited to serine (S), aminoisobutyric acid and an unnatural amino acid; Xaa₂ includes, but is not limited to arginine (R), aminoisobutyric acid and an unnatural amino acid; Xaa₃ includes, but is not limited to glutamic acid (E), aminoisobutyric acid and an unnatural amino acid; Xaa₄ includes, but is not limited to phenylalanine (F), glutamine (Q), alanine (A), aminoisobutyric acid and an unnatural amino acid; Xaa₅ includes, but is not limited to glycine (G), aminoisobutyric acid and an unnatural amino acid; Xaa₆ includes, but is not limited to phenylalanine (F), leucine (E), aminoisobutyric acid, 2-aminobutyric acid, and an unnatural amino acid, or is absent; Xaa₇ includes, but is not limited to glutamine (Q), aminoisobutyric acid and an unnatural amino acid, or is absent; Xaa₈ includes, but is not limited to phenylalanine (F), aminoisobutyric acid and an unnatural amino acid, or is absent; and Xaa₉ includes, but is not limited to aminoisobutyric acid and an unnatural amino acid, or is absent.

The term “cross-linker” or grammatical variations thereof as used herein refers to the intramolecular connection (also referred as staple) of two peptides domains (e.g., two loops of a helical peptide). When the peptide has a helical secondary structure, the cross-linker is a macrocyclic ring, which is exogenous (not part of) core or inherent (non-cross-linked) helical peptide structure. The macrocyclic ring may comprise an all-hydrocarbon linkage ring and incorporates the side chains linked to the α-carbon of at least two amino acids of the peptide. The size of the macrocyclic ring is determined by the number helical peptide amino acids in the ring and the number of carbon groups in the moieties connecting the α-carbon of the at least two amino acids of the peptide. The cross-linked peptide has at least one cross-linker. In various examples, the cross-linked peptide has 1, 2 or 3 cross linkers.

A cross-linked peptide (i.e. stapled peptide) is a peptide comprising a selected number of standard (i.e. natural) or non-standard (non-natural or unnatural or synthetic) amino acids, further comprising at least two moieties capable of undergoing reaction to promote carbon-carbon bond formation, that has been contacted with a reagent to generate at least one cross-link between the at least two moieties, which modulates, for example, peptide stability. The cross-linked peptide may comprise more than one, that is multiple (two, three, four, five, six, etc.) cross-links.

Any cross-linker known in the art can be used. Exemplary cross-linkers can include but are not limited to, hydrocarbon linkage, one or more of an ether, thioether, ester, amine, or amide moiety. In some cases, a naturally occurring amino acid side chain can be incorporated into the cross-linker. For example, a cross-linker can be coupled with a functional group such as the hydroxyl in serine, the thiol in cysteine, the primary amine in lysine, the acid in aspartate or glutamate, or the amide in asparagine or glutamine. Accordingly, it is possible to create a cross-link using naturally occurring amino acids rather than using a cross-linker that is made by coupling two non-naturally occurring amino acids. It is also possible to use a single non-naturally occurring amino acid together with a naturally occurring amino acid. In one example, there is provided a peptide as disclosed herein wherein the natural amino acid in the position to be cross-linked (i.e. the naturally occurring amino acid that is used to create the cross-linker) is replaced by an olefin-bearing unnatural amino acid. In a further example, the peptide as described above may comprise at least one two peptide cross linkers. Additionally, the peptide as described above is characterized by the presence of a first unnatural amino acid at the position Xaa₁, Xaa₂, Xaa₃ or Xaa₄ wherein the unnatural amino acid side chain cross-links to the side chain of a second unnatural amino acid at position Xaa₅, Xaa₆, Xaa₇, Xaa₈ or Xaa₉. In one example, the cross-linker of the peptide as described herein may comprise a hydrocarbon linkage.

In one example, the hydrocarbon linkage is an oleifinic group. The term “olefin” and grammatical variations thereof (also called alkene or alkenyl for a group) as used herein denotes a monovalent group derived from a straight- or branched-chain hydrocarbon moiety having at least one carbon-carbon double bond by the removal of a single hydrogen atom. The alkenyl moiety contains the indicated number of carbon atoms. For example, C₂-C₁₀ indicates that the group may have from 2 to 10 (inclusive) carbon atoms in it. The term “lower alkenyl” refers to a C₂-C₈ alkenyl chain. In the absence of any numerical designation, “alkenyl” is a chain (straight or branched) having 2 to 20 (inclusive) carbon atoms in it.

In certain embodiments, the olefinic group employed in the invention contains 2-20 carbon atoms. In some embodiments, the olefin group employed in the invention contains 2-15 carbon atoms. In another embodiment, the olefin group employed contains 2-10 carbon atoms. In still other embodiments, the olefin group contains 2-8 carbon atoms. In yet other embodiments, the olefinic group contains 2-5 carbons, or 2, 3, 4, 5, 6, 7 or 8 carbons.

Olefinic groups include, for example, ethenyl, propenyl, butenyl, 1-methyl-2-buten-1-yl, and the like, which may bear one or more substituents. Olefinic group substituents include, but are not limited to, any of the substituents described herein, that result in the formation of a stable moiety. Examples of substituents include, but are not limited to, the following groups: aliphatic, alkyl, olefinic, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, leteroalkylthioxy, arylthioxy, heteroarylthioxy, acyloxy, and the like, each of which may or may not be further substituted.

The compounds, proteins, or peptides of the present invention (e.g., amino acids, peptides and proteins) may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

It will be appreciated that the compounds of the present invention, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include substitution with all permissible substituents of organic compounds, any of the substituents described herein.

For example, the substituents include, but are not limited to, the following groups: aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, and halo and any combination thereof including, but not limited to, the following groups: aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, that result in the formation of a stable moiety. The present invention contemplates any and all such combinations in order to arrive at a stable substituent/moiety. For purposes of this invention, heteroatoms such as nitrogen may have hydrogen substituents and/or any suitable substituent as described herein which satisfy the valences of the heteroatoms and results in the formation of a stable moiety.

As used herein, substituent names which end in the suffix “-ene” refer to a biradical derived from the removal of two hydrogen atoms from the substitutent. Thus, for example, acyl is acylene; alkyl is alkylene; alkenyl is alkenylene; alkynyl is alkynylene; heteroalkyl is heteroalkylene, heteroalkenyl is heteroalkenylene, heteroalkynyl is heteroalkynylene, aryl is arylene, and heteroaryl is heteroarylene.

The term “aliphatic,” as used herein, includes both saturated and unsaturated, nonaromatic, straight chain (i.e., unbranched), branched, acyclic, and cyclic (i.e., carbocyclic) hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “aliphatic” is used to indicate those aliphatic groups (cyclic, acyclic, substituted, unsubstituted, branched or unbranched) having 1-20 carbon atoms or 1, 2, 3, 4, 5, 6, 7, 8, 9, 10, 11, 12, 13, 14, 15, 16, 17, 18, 19 or 20 carbon atoms.

The term “alkyl,” as used herein, refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-20 carbon atoms or 1, 2, 3, 4, 5, 6, 7, 8, 9, 10, 11, 12, 13, 14, 15, 16, 17, 18, 19 or 20 carbon atoms. In another embodiment, the alkyl group employed contains 1-15 carbon atoms. In another embodiment, the alkyl group employed contains 1-10 carbon atoms. In another embodiment, the alkyl group employed contains 1-8 carbon atoms. In another embodiment, the alkyl group employed contains 1-5 carbon atoms. For example, C₁-C₁₀ indicates that the group may have from 1 to 10 (inclusive) carbon atoms in it. In the absence of any numerical designation, “alkyl” is a chain (straight or branched) having 1 to 20 (inclusive) carbon atoms in it.

The term “alkylene,” as used herein, refers to a biradical derived from an alkyl group, as defined herein, by removal of two hydrogen atoms and thus refers to a divalent alkyl. Alkylene groups may be cyclic or acyclic, branched or unbranched, substituted or unsubstituted.

The term “alkenylene,” as used herein, refers to a biradical derived from an alkenyl group, as defined herein, by removal of two hydrogen atoms. Alkenylene groups may be cyclic or acyclic, branched or unbranched, substituted or unsubstituted.

The term “alkynyl,” as used herein, refers to a monovalent group derived from a straight- or branched-chain hydrocarbon having at least one carbon-carbon triple bond by the removal of a single hydrogen atom. In certain embodiments, the alkynyl group employed in the invention contains 2-20 carbon atoms or 1, 2, 3, 4, 5, 6, 7, 8, 9, 10, 11, 12, 13, 14, 15, 16, 17, 18, 19 or 20 carbon atoms. In some embodiments, the alkynyl group employed in the invention contains 2-15 carbon atoms. In another embodiment, the alkynyl group employed contains 2-10 carbon atoms. In still other embodiments, the alkynyl group contains 2-8 carbon atoms. In still other embodiments, the alkynyl group contains 2-5 carbon atoms.

The term “alkynylene,” as used herein, refers to a biradical derived from an alkynylene group, as defined herein, by removal of two hydrogen atoms. Alkynylene groups may be cyclic or acyclic, branched or unbranched, substituted or unsubstituted.

The term “amino,” as used herein, refers to a group of the formula (—NH₂). A “substituted amino” refers either to a mono-substituted amine (—NHR^{h}) of a disubstituted amine (—NR^{h}₂), wherein the R^{h} substituent is any substitutent as described herein that results in the formation of a stable moiety. For example, the substituent includes, but is not limited, to the following groups: a suitable amino protecting group; aliphatic, alkyl, alkenyl, alkynyl, heteroaliphatic, heterocyclic, aryl, heteroaryl, acyl, amino, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted. In certain embodiments, the R^{h} substituents of the di-substituted amino group (—NR^{h}₂) form a 5- to 6-membered hetereocyclic ring.

The terms “halo” and “halogen” as used herein refer to an atom selected from fluorine (fluoro, —F), chlorine (chloro, —Cl), bromine (bromo, —Br), and iodine (iodo, —I).

The term. “cycloalkyl” as employed herein includes saturated and partially unsaturated cyclic hydrocarbon groups having 3 to 12 carbons, preferably 3 to 8 carbons, more preferably 3 to 6 carbons, and 3, 4, 5, 6, 7, 8, 9, 10, 11, or 12 carbon atoms wherein the cycloalkyl group additionally may be optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S. For example, the heteroaryl may comprise carbon atoms and 1-3, 1-6, or 1-9 heteroatoms of N, O, or S if monocyclic, bicyclic, or tricyclic, respectively, wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S. For example, the heterocyclyl may comprise carbon atoms and 1-3, 1-6, or 1-9 heteroatoms of N, O, or S if monocyclic, bicyclic, or tricyclic, respectively, wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like.

The term “hydroxy,” or “hydroxyl,” as used herein, refers to a group of the formula (—OH). A “substituted hydroxyl” refers to a group of the formula (—OR¹), wherein R1 can be any substitutent which results in a stable moiety, as for example a suitable hydroxyl protecting group; aliphatic, alkyl, alkenyl, alkynyl, heteroaliphatic, heterocyclic, aryl, heteroaryl, acyl, nitro, alkylaryl, arylalkyl, and the like, each of which may or may not be further substituted.

The term “oxo,” as used herein, refers to a group of the formula (═O).

The term “thio,” or “thiol,” as used herein, refers to a group of the formula (—SH). A “substituted thiol” refers to a group of the formula (—SR1), wherein Rr can be any substituent that results in the formation of a stable moiety, as for example a suitable thiol protecting group; aliphatic, alkyl, alkenyl, alkynyl, heteroaliphatic, heterocyclic, aryl, heteroaryl, acyl, sulfinyl, sulfonyl, cyano, nitro, alkylaryl, arylalkyl, and the like, each of which may or may not be further substituted.

The term “substituents” refers to a group “substituted” as described above on an alkyl, cycloalkyl, aryl, heterocyclyl, or heteroaryl group at any atom of that group. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, and cyano groups.

The term “amino acid” refers to a molecule containing both an amino group and a carboxyl group. Amino acids include alpha-amino acids and beta-amino acids, the structures of which are depicted below. In certain embodiments, an amino acid is an alpha amino acid.

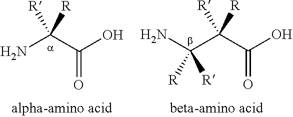

Suitable amino acids are known to the person skilled in the art and include, without limitation, natural alpha-amino acids such as D- and L-isomers of the 20 common naturally occurring alpha-amino acids found in peptides, that is, in one-letter code, A, R, N, C, D, Q, E, G, H, I, L, K, M, F, P, S, T, W, Y, and V, unnatural alpha-amino acids, natural beta-amino acids as for example, beta-alanine, and unnatural beta-amino acids. Amino acids known in the art (both naturally occurring and synthetic) which can be used for the peptides and/or modified peptides referred to herein (e.g. also for “*” or “Xaa”) can include, but are not limited to 2-aminoadipic acid (Aad), aminobutyric acid (Abu), aminobenzoic acid (Abz), aminocyclohexanoic acid (Ac6c), aminocyclopentanoic acid (Ac5c), aminocyclopropanoic acid (Ac3c), aminodecanoic acid (Adc), aminoundecanoic acid (Ado), aminohexanoic acid (Ahx), aminoisobutyric acid (Aib), alanine (Ala), alloisoleucine (Alle), allothreonine (aThr), aminomethylbenzoic acid (Amb), aminomethylcyclohexanoic acid (Amc), 2-amino-2-thiazolidine-4-carboxylic acid, aminononanoic acid, aminooctanoic acid, aminopentanoic acid (Avl), arginine (Arg), asparagine (Asn), aspartic acid (Asp), aminoundecanoic acid, aminovaleric acid, biphenylalanine, benzoylphenyl alanine, carnitine, 4-cyano-2-aminobutyric acid, 3-cyano-2-aminopropionic acid, cyclohexylalanine, cyclohexylglycine, citruline (Cit), cysteine (Cys), cystine, 2,4-diaminobutyric acid (A2bu), 2,3-diaminopropionic acid (A2pr), diethyl glycine, dihydrotryptophan, diaminobenzoic acid, dipropylglycine, 2,3-diaminopropionic acid, 2,3-didehydroalanine (Dha), (Z)-2,3-didehydroaminobutyric acid (Dhb), erythro-3-hydroxyaspartic acid (HyAsp), 2-aminobutyric acid (Abu), dolaproine (Dap), dolaisoluine (Dil), dolaisovaline (Dov), Hiv, methyl valine (MeVal), 3-amino-6-octyneoic acid (Doy), dolaphenine (Doe), dolahexanoic acid (Dhex) 2-methyl-3-aminoisocaproic acid (Dml, dolamethylleuine), 2-amino-4-phenylisovaleric acid (Dpv, dolaphenvaline), diethylglycine, dihydrotryptophan, gamma-carboxyglutamic acid, glutamine (Gin), glutamic acid (Glu), glycine (Gly), histidine (His), homoarginine, homocysteine (Hey), homophenylalanine, homoserine (Hse), homoserinelactone (Hsl), homotyrosine, hydroxylysine (Hyl), hydroxyproline (Hyp), 2-indolinecarboxylic acid, 2-indanylglycine, isoglutamine (iGIn), isoleucine (He), indoleglycine, isonipecotic acid, isovaline (Iva), leucine (Leu), lysine (Lys), /3-mercapto-3,/3-cyclopentamethylenepropanoic acid, methionine (Met), methionine S-oxide (Met(O)), muramicacid (Mur), napthylalanine, neuraminicacid (Neu), norleucine (Nle), norvaline (Nva), octahydroindolecarboxylic acid, ornithine (Orn), pyridylalanine, penicillamine, pyroglutamic acid, phenylalanine (Phe), Cₐ-Me-L-Phenylalanine, phenylglycine, phosphoserine (Ser(P)), pipecolic acid, 4-phosphomethylphenylalanine, propargylglycine, proline (Pro), putrescine, sarcosine (Sar), serine (Ser), statine (Sta), statine analogs, taurine (Tau), thiazolidinecarboxylic acid, tetrahydroisoquinoline-3-carboxylic acid, tert-leucine, threonine (Thr), thyroxine (Thx), tryptophan (Trp), tyrosine (Tyr), 3,5-diiodotyrosine (Tyr(I₂)), valine (Val) and AEEA. Abbreviations for amino acids, as used herein, are in accordance with the IUPAC guidelines on nomenclature.

Amino acids used in the construction of peptides of the present invention may be prepared by organic synthesis, or obtained by other routes, such as for example, degradation of or isolation from a natural source. In certain examples of the present invention, the formula —[X_{AA}]— corresponds to the natural and/or unnatural amino acids having the following formulae:

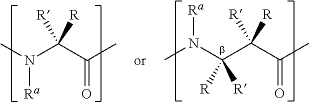

wherein R and R′ correspond a suitable amino acid side chain, as defined below, and R^{a} is as defined below.

There are many known unnatural amino acids any of which may be included in the peptides of the present invention. Some examples of unnatural amino acids are (S)-2-(4′-pentenyl)alanine, (R)-2-(4′-pentenyl)alanine, (S)-2-(7′-octenyl)alanine, (R)-2-(7′-octenyl)alanine and any one of the aforementioned amino acids with varied length. Other examples include but are not limited to 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; —CN; -halo; —NO₂; CH₃), disubstituted phenylalanines, substituted tyrosines (e.g., further substituted with —C(═O)C₆H5; —CF₃; —CN; -halo; —NO₂; CH₃), and statine. Additionally, the amino acids suitable for use in the present invention may be derivatized to include amino acid residues that are hydroxylated, phosphorylated, sulfonated, acylated, and glycosylated, to name a few.

The term “amino acid side chain” refers to a group or moiety attached to the alpha- or beta-carbon of an amino acid. A “suitable amino acid side chain” includes, but is not limited to, any of the suitable amino acid side chains as known in the art.

For example, suitable amino acid side chains include methyl (as the alpha-amino acid side chain for alanine is methyl), 4-hydroxyphenylmethyl (as the alpha-amino acid side chain for tyrosine is 4-hydroxyphenylmethyl) and thiomethyl (as the alpha-amino acid side chain for cysteine is thiomethyl), etc. Other non-naturally occurring amino acid side chains are also included, for example, those that occur in nature (e.g., an amino acid metabolite) or those that are made synthetically (e.g., an alpha di-substituted amino acid).

A “peptide” or “polypeptide” comprises a polymer of amino acid residues linked together by peptide (amide) bonds. The term(s), as used herein, refers to proteins, polypeptides, and peptide of any size, structure, or function. Typically, a peptide or polypeptide will be at least three amino acids long. A peptide or polypeptide may refer to an individual protein or a collection of proteins. Inventive proteins preferably contain only natural amino acids, although non-natural amino acids that is, compounds that do not occur in nature but that can be incorporated into a polypeptide chain and/or amino acid analogs as are known in the art may alternatively be employed. Also, one or more of the amino acids in a peptide or polypeptide may be modified, for example, by the addition of a chemical entity such as a carbohydrate group, a hydroxyl group, a phosphate group, a farnesyl group, an isofarnesyl group, a fatty acid group, a linker for conjugation, functional ization, or other modification, etc. A peptide or polypeptide may also be a single molecule or may be a multi-molecular complex, such as a protein. A peptide or polypeptide may be just a fragment of a naturally occurring protein or peptide. A peptide or polypeptide may be naturally occurring, recombinant, or synthetic, or any combination thereof. As used herein “dipeptide” refers to two covalently linked amino acids.

As used herein, when two entities are “associated with” one another they are linked by a direct or indirect covalent or non-covalent interaction. In certain embodiments, the association is covalent and the entities are “conjugated” to one another. In other embodiments, the association is non-covalent. Non-covalent interactions include hydrogen bonding, van der Waals interactions, hydrophobic interactions, magnetic interactions, electrostatic interactions, etc. An indirect covalent interaction is when two entities are covalently associated through a linker.

As used herein, when two entities are “conjugated” to one another they are linked by a direct or indirect covalent interaction. An indirect covalent interaction is when two entities are covalently connected, optionally through a linker.

In one example, there is provided the peptide as described herein, wherein the first amino acid at position Xaa₁, Xaa₂, Xaa₃ or Xaa₄ that cross-links the second amino acid to position Xaa₅, Xaa₆, Xaa₈ or Xaa₉ in the position of the peptide cross-linker are both olefin-bearing unnatural amino acids.

In one example, there is provided a peptide as disclosed herein, wherein the olefin-bearing unnatural amino acid is selected from the group consisting of (S)-2-(4′-pentenyl)alanine, (R)-2-(4′-pentenyl)alanine, (S)-2-(7′-octenyl)alanine, (R)-2-(7′-octenyl)alanine and any one of the aforementioned amino acids with varied length.

In one embodiment, the peptide of the present invention, may comprise the cross-linker that is a cysteine bridge or a Lys-Asn (Lysine-Asparagine) linker. In some embodiments, the peptide can comprise at least one capping group at the N-terminus and/or the C-terminus. The capping group at the N-terminus of the modified eIF4G1 peptide usually has hydrogen atoms able to form hydrogen bonds or having a negative charge at the N-terminus to match with the helix dipole, a non-peptidic group or a mimic of an amino acid side chain. Suitable N-terminal capping groups include acyl such as acetyl, or N-succinate. The C-terminal capping group usually has hydrogen atoms able to form hydrogen bonds or having a positive charge at the C-terminus to match with the helix dipole. A suitable C-terminal capping group is an amide group or NH₂. In one example, there is provided the peptide as described above and herein, wherein the C-terminus of the peptide is amidated. In another example, there is provided the peptide as described above and herein, wherein the N-terminus of the peptide is acetylated. To functionalize the peptide of the invention and improve its biological activity, it is proved the peptide as described herein, wherein the peptide is modified to include but is not limited to one or more ligands hydroxyl, phosphate, amine, amide, sulphate, sulphide, a biotin moiety, a carbohydrate moiety, a fatty acid-derived acid group, a fluorescent moiety, a chromophore moiety, a radioisotope, a PEG linker, an affinity label, a targeting moiety, an antibody, a cell penetrating peptide and a combination of the aforementioned ligands.

In one example the peptide described herein is not or does not comprise the amino acid sequence KKRYSREFLLGF.

In one example, there is provided a peptide as described above and herein, wherein the peptide comprises formula I:

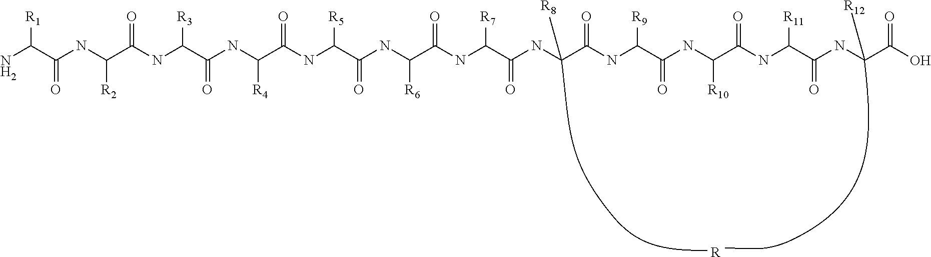

wherein: R₁ and R₂ are —(CH₂)₄—NH₂ [K]; R₃ is —(CH₂)₃—NH—C(NH₂)₂ [R]; R₄ is —CH₂-Phenyl-OH [Y]; R₅ is —CH₂—OH [S]; R₆ is —(CH₂)₃—NH—C(NH₂)₂ [R]; R₇ is —(CH₂)₂C(O)OH [E], or aminoisobutyric acid; R₈ and R₁₂ are independently H, a C₁ to C₁₀ alkyl, alkenyl, alkynyl, arylalkyl, cycloalkylalkyl, heteroarylalkyl, or heterocyclylalkyl; R₉ and R₁₀ are —CH₂CH(CH₃)₂ [L]; and R₁₁ is —H [G] or aminoisobutyric acid; and wherein R is anyone of alkyl, alkenyl, alkynyl, or [R′—K—R″]n; each of which is substituted with 0, 1, 2, 3, 4, 5, or 6 R₁₃; R′ and R″ are independently alkylene, alkynylene or alkynylene; each R₁₃ is independently halo, alkyl, OR₁₄, N(R₁₄)₂, SR₁₄, SOR₁₄, SO₂R₁₄, CO₂R₁₄, R₁₄, a fluorescent moiety, or a radioisotope; K is independently O, S, SO, SO₂, CO, CO₂, or CONR₁₄; each R14 is independently H, alkyl, or a therapeutic agent; n is an integer from 0, 1, 2, 3 or 4.

In a further example, there is provided the peptide described above, wherein R₈ and R₁₂ are independently H or a C₁ to C₆ alkyl. In another example, there is provided the peptide described above, wherein R₈ and R₁₂ are each —CH₃ [A] and R is a 4′-cyclooctenyl

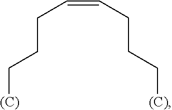

(sTIP-02) cross-linking the α-carbon of the two unnatural amino-acids. sTIP-02 is described in more detail in Table 2, for example. The Kd of sTIP-02 is in the nanomolar range.

The cross-link may be obtained by chemical reactions known in the art. For example, the cross-link is obtained by olefin ring-closing metathesis in the presence of a Grubbs catalyst, thereby forming a 4′-cyclooctenyl as described above and in the figure above.

Thus, there is provided the peptide described above, wherein R is obtained by cross-linking the pentenyl side chains having the S stereochemistry of (S)-2-(4′-pentenyl)alanine at position Xaa₂ (i; R₈ is CH₃) and at position Xaa₄ at a position four amino acids apart (i+4; R₁₂ is CH₃), wherein the pentenyl side chains of both Xaa₂ and Xaa₄ are linked to the α-carbon of the two unnatural amino-acids and are on the same side of the α-helix (sTIP-02).

In another example, there is provided a peptide of the present invention, wherein the peptide comprises formula II:

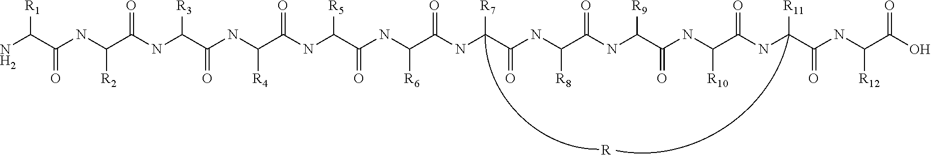

wherein: R₁ and R₂ are —(CH₂)₄—NH₂ [K]; R₃ is —(CH₂)₃—NH—C(NH₂)₂ [R]; R₄ is —CH₂-Phenyl-OH [Y]; R₅ is —CH₂—OH [S]; R₆ is —(CH₂)₃—NH—C(NH₂)₂ [R]; R₇ and R₁₁ are independently H, a C₁ to C₁₀ alkyl, alkenyl, alkynyl, arylalkyl, cycloalkylalkyl, heteroarylalkyl, or heterocyclylalkyl; R₈ is benzyl [F], or —(CH₂)₂—C(O)NH₂ [Q], or —CH₃ [A], or aminoisobutyric acid; R₉ and R₁₀ are —CH₂CH(CH₃)₂ [L]; and R₁₂ is benzyl [F], or —CH₂CH(CH₃)₂ [L], or aminoisobutyric acid; and wherein R is anyone of alkyl, alkenyl, alkynyl, or [R′—K—R″]n; each of which is substituted with 0, 1, 2, 3, 4, 5, or 6 R₁₃; R′ and R″ are independently alkylene, alkenylene or alkynylene; each R₁₃ is independently halo, alkyl, OR₁₄, N(R₁₄)₂, SR₁₄, SOR₁₄, SO₂R₁₄, CO₂R₁₄, —R₁₄, a fluorescent moiety, or a radioisotope; K is independently O, S, SO, SO₂, CO, CO₂, or CONR₁₄; each R14 is independently H, alkyl, or a therapeutic agent; n is an integer from 0, 1, 2, 3 or 4.

Accordingly, in a further example, there is provided a peptide of the present invention as described above, wherein R₇ and R₁₁ are independently H or a C₁ to C₆ alkyl. In another example, the peptide described has a CH₃ (methyl) at each one of position R₇ and R₁₁; a 4′-cyclooctenyl at position R is and; R₈ is benzyl [F] and R₁₂ is independently benzyl [F] (also described as TIP-01 in Table 2 below) or 2-aminobutyric acid (sTIP-01F^{12&}), or; R₈ is —(CH₂)₂—C(O)NH₂ [Q] and R₁₂ is —CH₂CH(CH₃)₂ [L] (sTIP-04) or; R₈ is methyl [A] and R₁₂ is benzyl [F] (sTIP-01 F^{8A}).

In another example, there is provided a peptide of the present invention, wherein the peptide comprises formula III:

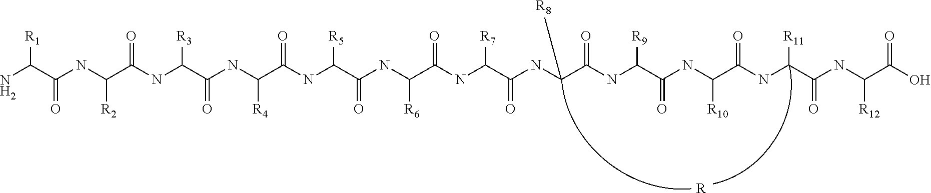

Wherein R₁ and R₂ are —(CH₂)₄—NH₂ [K]; R₃ is —(CH₂)₃—NH—C(NH₂)₂ [R]; R₄ is —CH₂-Phenyl-OH [Y]; R₅ is —CH₂—OH [S]; R₆ is —(CH₂)₃—NH—C(NH₂)₂ [R]; R₇ is —(CH₂)₂C(O)OH [E], or aminoisobutyric acid; R₈ and R₁₁ are independently H, a C₁ to C₁₀ alkyl, alkenyl, alkynyl, arylalkyl, cycloalkylalkyl, heteroarylalkyl, or heterocyclylalkyl; R₉ and R₁₀ are —CH₂CH(CH₃)₂ [L]; and R₁₂ is benzyl [F], or —CH₂CH(CH₃)₂ [L], or aminoisobutyric acid; and wherein R is anyone of alkyl, alkenyl, alkynyl, or [R′—K—R″]n; each of which is substituted with 0, 1, 2, 3, 4, 5, or 6 R₁₃; R′ and R″ are independently alkylene, alkenylene or alkynylene; each R₁₃ is independently halo, alkyl, OR₁₄, N(R₁₄)₂, SR₁₄, SOR₁₄, SO₂R₁₄, CO₂R₁₄, R₁₄, a fluorescent moiety, or a radioisotope; K is independently O, S, SO, SO₂, CO, CO₂, or CONR₁₄; each R14 is independently H, alkyl, or a therapeutic agent; n is an integer from 0, 1, 2, 3 or 4.

In another example, there is provided the peptide as disclosed above and herein, wherein R₈ and R₁₁ are independently H or a C₁ to C₆ alkyl. In another example, there is provided the peptide as described above, wherein R₈ and R₁₁ are each —CH₃ [A] and R is a 4′-cyclooctenyl (sTIP-03) cross-linking the α-carbon of the two unnatural amino-acids.

In one example, there is provided a peptide as disclosed above and herein, wherein the peptide comprises formula IV:

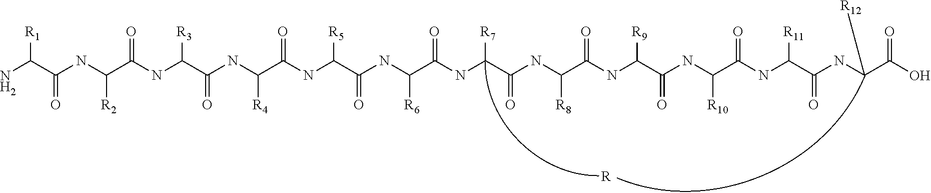

wherein: R₁ and R₂ are —(CH₂)₄—NH₂ [K]; R₃ is —(CH₂)₃—NH—C(NH₂)₂ [R]; R₄ is —CH₂-Phenyl-OH [Y]; R₅ is —CH₂—OH [S]; R₆ is —(CH₂)₃—NH—C(NH₂)₂ [R]; R₇ and R₁₂ are independently H, a C₁ to C₁₀ alkyl, alkenyl, alkynyl, arylalkyl, cycloalkylalkyl, heteroarylalkyl, or heterocyclylalkyl; R₈ is benzyl [F], or —(CH₂)₂—C(O)NH₂ [Q], or —CH₃ [A], or aminoisobutyric acid; R₉ and R₁₀ are —CH₂CH(CH₃)₂ [L]; and R₁₁ is —H [G] or aminoisobutyric acid; and wherein R is anyone of alkyl, alkenyl, alkynyl, or [R′—K—R″]n; each of which is substituted with 0, 1, 2, 3, 4, 5, or 6 R₁₃; R′ and R″ are independently alkylene, alkenylene or alkynylene; each R₁₃ is independently halo, alkyl, OR₁₄, N(R₁₄)₂, SR₁₄, SOR₁₄, SO₂R₁₄, CO₂R₁₄, R₁₄, a fluorescent moiety, or a radioisotope; K is independently O, S, SO, SO₂, CO, CO₂, or CONR₁₄; each R14 is independently H, alkyl, or a therapeutic agent; n is an integer from 0, 1, 2, 3 or 4.

In a further example, there is provided the peptide as described above, wherein R₇ and R₁₂ are independently H or a C₁ to C₆ alkyl.

In one example, there is provided a peptide of the present invention, wherein the peptide comprises formula V:

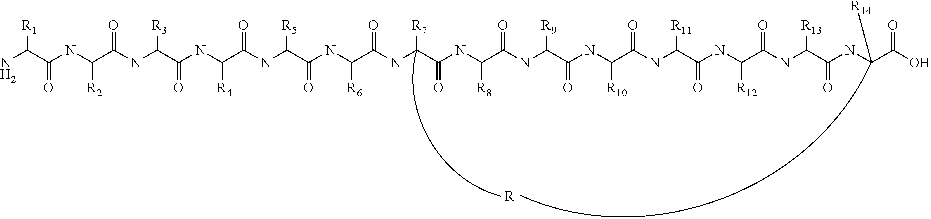

Wherein R₁ and R₂ are —(CH₂)₄—NH₂ [K]; R₃ is —(CH₂)₃—NH—C(NH₂)₂ [R]; R₄ is —CH₂-Phenyl-OH [Y]; R₅ is —CH₂—OH [S]; R₆ is —(CH₂)₃—NH—C(NH₂)₂ [R]; R₇ and R₁₄ are independently H or a C₁ to C₁₀ alkyl, alkenyl, alkynyl, arylalkyl, cycloalkylalkyl, heteroarylalkyl, or heterocyclylalkyl; R₈ is benzyl [F], or —(CH₂)₂—C(O)NH₂ [Q], or —CH₃ [A], or aminoisobutyric acid; R₉ and R₁₀ are —CH₂CH(CH₃)₂ [L]; and R₁₁ is —H [G] or aminoisobutyric acid; R₁₂ is benzyl [F]; R₁₃ is —(CH₂)₂—C(O)NH₂ [Q]; R₁₄ is benzyl [F]; and wherein R is anyone of alkyl, alkenyl, alkynyl, or [R′—K—R″]n; each of which is substituted with 0, 1, 2, 3, 4, 5, or 6 R₁₆; R′ and R″ are independently alkylene, alkenylene or alkynylene; each R₁₆ is independently halo, alkyl, OR₁₇, N(R₁₇)₂, SR₁₇, SOR₁₇, SO₂R₁₇, CO₂R₁₇, R₁₇, a fluorescent moiety, or a radioisotope; K is independently O, S, SO, SO₂, CO, CO₂, or CONR₁₇; each R₁₇ is independently H, alkyl, or a therapeutic agent; n is an integer from 0, 1, 2, 3 or 4.

In another example, there is provided the peptide as disclosed above and herein, wherein R₇ and R₁₄ are independently H or a C₁ to C₆ alkyl. In another example, there is provided the peptide as described above, wherein R₇ and R₁₄ are each —CH₃ [A] and R is a 4′-cyclooctenyl (sTIP-05) cross-linking the α-carbon of the two unnatural amino-acids.

In one example, there is provided a peptide of the present invention, wherein the peptide comprises formula VI:

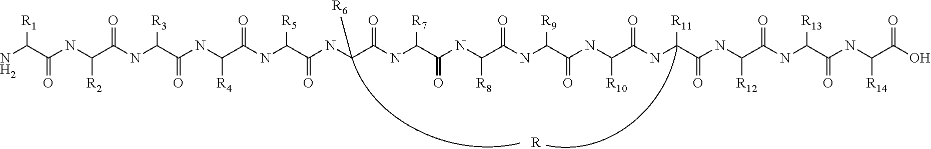

Wherein R₁ and R₂ are —(CH₂)₄—NH₂ [K]; R₃ is —(CH₂)₃—NH—C(NH₂)₂ [R]; R₄ is —CH₂-Phenyl-OH [Y]; R₅ is —CH₂—OH [S]; R₆ and R₁₁ are independently H or a C₁ to C₁₀ alkyl, alkenyl, alkynyl, arylalkyl, cycloalkylalkyl, heteroarylalkyl, or heterocyclylalkyl; R₇ is —(CH₂)₂C(O)OH [E], or aminoisobutyric acid; R₈ is benzyl [F], or —(CH₂)₂—C(O)NH₂ [Q], or —CH₃ [A], or aminoisobutyric acid; R₉ and R₁₀ are —CH₂CH(CH₃)₂ [L]; R₁₂ is benzyl [F]; R₁₃ is —(CH₂)₂—C(O)NH₂ [Q]; R₁₄ is benzyl [F]; and wherein R is anyone of alkyl, alkenyl, alkynyl, or [R′—K—R″]n; each of which is substituted with 0, 1, 2, 3, 4, 5, or 6 R₁₆; R′ and R″ are independently alkylene, alkenylene or alkynylene; each R₁₆ is independently halo, alkyl, OR₁₇, N(R₁₇)₂, SR₁₇, SOR₁₇, SO₂R₁₇, CO₂R₁₇, R₁₇, a fluorescent moiety, or a radioisotope; K is independently O, S, SO, SO₂, CO, CO₂, or CONR₁₇; each R₁₇ is independently H, alkyl, or a therapeutic agent; n is an integer from 0, 1, 2, 3 or 4.

In another example, there is provided the peptide as disclosed above and herein, wherein R₆ and R₁₁ are independently H or a C₁ to C₆ alkyl. In another example, there is provided the peptide as described above, wherein R₆ and R₁₁ are each —CH₃ [A] and R is a 4′-cyclooctenyl (sTIP-06) cross-linking the α-carbon of the two unnatural amino-acids.

In one example, there is provided a peptide of the present invention, wherein the peptide comprises formula VII:

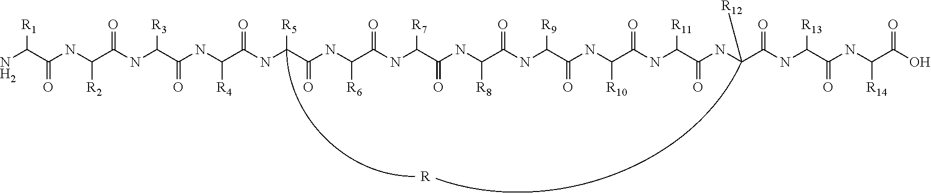

Wherein R₁ and R₂ are —(CH₂)₄—NH₂ [K]; R₃ is —(CH₂)₃—NH—C(NH₂)₂ [R]; R₄ is —CH₂-Phenyl-OH [Y]; R₅ and R₁₂ are independently H or a C₁ to C₁₀ alkyl, alkenyl, alkynyl, arylalkyl, cycloalkylalkyl, heteroarylalkyl, or heterocyclylalkyl; R₆ is —(CH₂)₃—NH—C(NH₂)₂ [R]; R₇ is —(CH₂)₂C(O)OH [E], or aminoisobutyric acid; R₈ is benzyl [F], or —(CH₂)₂—C(O)NH₂ [Q], or —CH₃ [A], or aminoisobutyric acid; R₉ and R₁₀ are —CH₂CH(CH₃)₂ [L]; R₁₁ is —H [G] or aminoisobutyric acid; R₁₃ is —(CH₂)₂—C(O)NH₂ [Q]; R₁₄ is benzyl [F]; and wherein R is anyone of alkyl, alkenyl, alkynyl, or [R′—K—R″]n; each of which is substituted with 0, 1, 2, 3, 4, 5, or 6 R₁₆; R′ and R″ are independently alkylene, alkenylene or alkynylene; each R₁₆ is independently halo, alkyl, OR₁₇, N(R₁₇)₂, SR₁₇, SOR₁₇, SO₂R₁₇, CO₂R₁₇, R₁₇, a fluorescent moiety, or a radioisotope; K is independently O, S, SO, SO₂, CO, CO₂, or CONR₁₇; each R₁₇ is independently H, alkyl, or a therapeutic agent; n is an integer from 0, 1, 2, 3 or 4.

In another example, there is provided the peptide as disclosed above and herein, wherein R₅ and R₁₂ are independently H or a C₁ to C₆ alkyl. In another example, there is provided the peptide as described above, wherein R₅ and R₁₂ are each —CH₃ [A] and R is a 4′-cyclooctenyl (sTIP-07) cross-linking the α-carbon of the two unnatural amino-acids.

In one example, there is provided a peptide of the present invention, wherein the peptide comprises formula VIII:

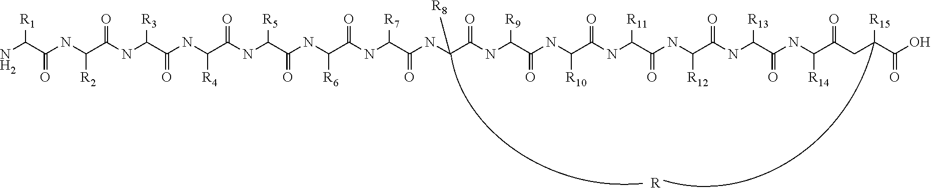

Wherein R₁ and R₂ are —(CH₂)₄—NH₂ [K]; R₃ is —(CH₂)₃—NH—C(NH₂)₂ [R]; R₄ is. —CH₂-Phenyl-OH [Y]; R₅ is —CH₂—OH [5]; R₆ is —(CH₂)₃—NH—C(NH₂)₂ [R]; R₇ is —(CH₂)₂C(O)OH [E], or aminoisobutyric acid; R₈ and R₁₅ are independently H or a C₁ to C₁₀ alkyl, alkenyl, alkynyl, arylalkyl, cycloalkylalkyl, heteroarylalkyl, or heterocyclylalkyl; R₉ and R₁₀ are —CH₂CH(CH₃)₂ [L]; R₁₁ is —H [G] or aminoisobutyric acid; R₁₂ is benzyl [F]; R₁₃ is —(CH₂)₂—C(O)NH₂ [Q]; R₁₄ is benzyl [F]; and wherein R is anyone of alkyl, alkenyl, alkynyl, or [R′—K—R″]n; each of which is substituted with 0, 1, 2, 3, 4, 5, or 6 R₁₆; R′ and R″ are independently alkylene, alkenylene or alkynylene; each R₁₆ is independently halo, alkyl, OR₁₇, N(R₁₇)₂, SR₁₇, SOR₁₇, SO₂R₁₇, CO₂R₁₇, R₁₇, a fluorescent moiety, or a radioisotope; K is independently O, S, SO, SO₂, CO, CO₂, or CONR₁₇; each R₁₇ is independently H, alkyl, or a therapeutic agent; n is an integer from 0, 1, 2, 3 or 4.

In another example, there is provided the peptide as disclosed above and herein, wherein R₈ and R₁₅ are independently H or a C₁ to C₆ alkyl. In another example, there is provided the peptide as described above, wherein R₈ and R₁₅ are each —CH₃ [A] and R is a 4′-cyclooctenyl (sTIP-08) cross-linking the α-carbon of the two unnatural amino-acids.

In addition, the present invention also provides a nucleic acid molecule encoding for a peptide serving as template for the peptide of the present invention. Since the degeneracy of the genetic code permits substitutions of certain codons by other codons which specify the same amino acid and hence give rise to the same protein, the invention is not limited to a specific nucleic acid molecule but includes all nucleic acid molecules comprising a nucleotide sequence coding for the peptides of the present invention. The peptides encoded by the nucleic acid molecule may be chemically or enzymatically modified to obtain the cross-linked peptides as described herein.

The nucleic acid molecule disclosed herein may comprise a nucleotide sequence encoding the peptide serving as template for the peptide of the present invention which can be operably linked to a regulatory sequence to allow expression of the nucleic acid molecule. A nucleic acid molecule such as DNA is regarded to be ‘capable of expressing a nucleic acid molecule or a coding nucleotide sequence’ or capable ‘to allow expression of a nucleotide sequence’ if it contains regulatory nucleotide sequences which contain transcriptional and translational information and such sequences are “operably linked” to nucleotide sequences which encode the polypeptide. An operable linkage is a linkage in which the regulatory DNA sequences and the DNA sequences sought to be expressed are connected in such a way as to permit gene sequence expression. The precise nature of the regulatory regions needed for gene sequence expression may vary from organism to organism, but shall, in general include a promoter region which, in prokaryotes, contains only the promoter or both the promoter which directs the initiation of RNA transcription as well as the DNA sequences which, when transcribed into RNA will signal the initiation of synthesis. Such regions will normally include non-coding regions which are located 5′ and 3′ to the nucleotide sequence to be expressed and which are involved with initiation of transcription and translation such as the TATA box, capping sequence and CAAT sequences. These regions can for, example, also contain enhancer sequences or translated signal and leader sequences for targeting the produced polypeptide to a specific compartment of a host cell, which is used for producing a peptide described above.

The nucleic acid molecule comprising the nucleotide sequence encoding the modified eIF4G1 peptide of the invention can be comprised in a vector, for example an expression vector. Such a vector can comprise, besides the above-mentioned regulatory sequences and a nucleic acid sequence which codes for a peptide as described above, a sequence coding for restriction cleavage site which adjoins the nucleic acid sequence coding for the peptide in 5′ and/or 3′ direction. This vector can also allow the introduction of another nucleic acid sequence coding for a protein to be expressed or a protein part. The expression vector preferably also contains replication sites and control sequences derived from a species compatible with the host that is used for expression. The expression vector can be based on plasmids well known to person skilled in the art such as pBR322, puC16, pBluescript and the like.

The vector containing the nucleic acid molecule can be transformed into host cells capable of expressing the genes. The transformation can be carried out in accordance with standard techniques. Thus, the invention is also directed to a (recombinant) host cell containing a nucleic acid molecule as defined above. In this context, the transformed host cells can be cultured under conditions suitable for expression of the nucleotide sequence encoding the peptide as described above. Host cells can be established, adapted and completely cultivated under serum free conditions, and optionally in media which are free of any protein/peptide of animal origin. Commercially available media such as RPMI-1640 (Sigma), Dulbecco's Modified Eagle's Medium (DMEM; Sigma), Minimal Essential Medium (MEM; Sigma), CHO-S-SFMII (Invitrogen), serum free-CHO Medium (Sigma), and protein-free CHO Medium (Sigma) are exemplary appropriate nutrient solutions. Any of the media may be supplemented as necessary with a variety of compounds, examples of which are hormones and/or other growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics, trace elements. Any other necessary supplements may also be included at appropriate concentrations that are known to those skilled in the art.

As used herein, “nucleic acid” refers to any acid in any possible configuration, such as linearized single stranded, double stranded or a combination thereof. Nucleic acids may include, but are not limited to DNA molecules (e.g., cDNA or genomic DNA), RNA molecules (e.g., mRNA), analogues of the DNA or RNA generated using nucleotide analogues or using nucleic acid chemistry, cDNA synthetic DNA, a copolymer of DNA and RNA, oligonucleotides, and PNA (protein nucleic acids). DNA or RNA may be of genomic or synthetic origin and may be single or double stranded. A respective nucleic acid may furthermore contain non-natural nucleotide analogues and/or be linked to an affinity tag or a label. As used herein, nucleotides include nucleoside mono-, di-, and triphosphates. Nucleotides also include modified-nucleotides, such as, but not limited to, phophorothioate nucleotides and deazapurine nucleotides and other nucleotide analogs.

The peptide, the isolated nucleic acid molecule or the vector as described herein and above can be formulated into compositions, for example pharmaceutical compositions, suitable for administration. Where applicable, a peptide of the present invention may be administered with a pharmaceutically acceptable carrier. A “carrier” can include any pharmaceutically acceptable carrier as long as the carrier can is compatible with other ingredients of the formulation and not injurious to the patient. Accordingly, pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

Therefore, the present invention also provides a pharmaceutical composition comprising a one or more peptide of the present invention.

A peptide as described above or pharmaceutical composition or medicament thereof can be administered in a number of ways depending upon whether local or systemic administration is desired and upon the area to be treated. In some embodiments, the peptide or the respective pharmaceutical composition thereof can be administered to the patient orally, or rectally, or transmucosally, or intestinally, or intramuscularly, or subcutaneously, or intramedullary, or intrathecally, or direct intraventricularly, or intravenously, or intravitreally, or intraperitoneally, or intranasally, or intraocularly.

The peptides themselves may be present in the compositions in any of a wide variety of forms. For example, two or more peptides may be merely mixed together or may be more closely associated through complexation, crystallization, or ionic or covalent bonding. The peptides of the invention can also encompass any pharmaceutically acceptable salts, esters, or salts of such esters, or any other compound, which, upon administration to an animal, including a human, is capable of providing the biologically active metabolite or residue thereof. Accordingly, also described herein is drawn to prodrugs and pharmaceutically acceptable salts of such pro-drugs, and other bioequivalents. The term “pharmaceutically acceptable salt” refers to physiologically and pharmaceutically acceptable salt(s) of the peptides as described above; i.e. salts that retain the desired biological activity of the peptide and do not impart undesired toxicological effects thereto. Examples of such pharmaceutically acceptable salts include but are not limited to (a) salts formed with cations such as sodium, potassium, ammonium, magnesium, calcium, polyamines such as spermine and spermidine, etc; (b) acid addition salts formed with inorganic acids, for example hydrochloric acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chorine, bromine, and iodine.

In some embodiments, the pharmaceutical composition as described above and herein may further comprise a therapeutic compound (or an agent or a molecule or a composition). A “therapeutic” compound as defined herein is a compound (or an agent or a molecule or a composition) capable of acting prophylactically to prevent the development of a weakened and/or unhealthy state; and/or providing a subject with a sufficient amount of the complex or pharmaceutical composition or medicament thereof so as to alleviate or eliminate a disease state and/or the symptoms of a disease state, and a weakened and/or unhealthy state. In one example, the therapeutic compound includes but is not limited to an apoptosis promoting compound, a chemotherapeutic compound or a compound capable of alleviating or eliminating cancer in a patient. Examples of apoptosis promoting compounds include but are not limited to Cyclin-dependent Kinase (CDK) inhibitors, Receptor Tyrosine Kinase (RTK) inhibitors, BCL (B-cell lymphoma) family BH3 (Bcl-2 homology domain 3)-mimetic inhibitors and Ataxia Telangiectasia Mutated (ATM) inhibitors.

In an example, the Cyclin-dependent Kinase (CDK) inhibitors include but are not limited to 2-(R)-(1-Ethyl-2-hydroxyethyl amino)-6-benzylamino-9-isopropylpurine (CYC202; Roscovitine; Seliciclib); 4-[[5-Amino-1-(2,6-difluorobenzoyl)-1H-1,2,4-triazol-3-yl]amino]benzenesulfonamide (JNJ-7706621); N-(4-piperidinyl)-4-(2,6-dichlorobenzoylamino)-1H-pyrazole-3-carboxamide (AT-7519); N-(5-(((5-(1,1-dimethylethyl)-2-oxazolyl)methyl)thio)-2-thiazolyl)-4-piperidinecarboxamide (SNS-032); 8,12-Epoxy-1H,8H-2,7b,12a-triazadibenzo(a,g)cyclonona(cde)triinden-1-one, 2,3,9,10,11,12-hexahydro-3-hydroxy-9-methoxy-8-methyl-10-(methylamino)-(UCN-01; 7-Hydroxystaurosporine; KRX-0601); N,1,4,4-tetramethyl-8-((4-(4-methylpiperazin-1-yl)phenyl)amino)-4,5-dihydro-1H-pyrazolo[4,3-h]quinazoline-3-carboxamide (PHA-848125; milciclib); 2-(2-chlorophenyl)-5,7-dihydroxy-8-[(3S,4R)-3-hydroxy-1-methylpiperidin-4-yl]chromen-4-one hydrochloride (flavopiridol; alvocidib); 6-acetyl-8-cyclopentyl-5-methyl-2-((5-(piperazin-1-yl)pyridin-2-yl)amino)pyrido[2,3-d]pyrimidin-7(8H)-one hydrochloride (PD 0332991); 4-(1-isopropyl-2-methyl-1H-imidazol-5-yl)-N-(4-(methylsulfonyl)phenyl)pyrimidin-2-amine (AZD5438); (S)-3-(((3-ethyl-5-(2-(2-hydroxyethyl)piperidin-1-yl)pyrazolo[1,5-a]pyrimidin-7-yl)amino)methyl)pyridine 1-oxide (Dinaciclib; SCH 727965); N-(4-Piperidinyl)-4-(2,6-dichlorobenzoylamino)-1H-pyrazole-3-carboxamide hydrochloride (AT-7519); and pharmaceutically acceptable salts thereof.

In another example, there is provided the pharmaceutical composition as described above, wherein the RTK inhibitors include but are not limited to N-[3-chloro-4-[(3-fluorophenyl)methoxy]phenyl]-6-[5-[(2-methylsulfonylethylamino)methyl]-2-furyl]quinazolin-4-amine (lapatinib); N1′-[3-fluoro-4-[[6-methoxy-7-(3-morpholinopropoxy)-4-quinolyl]oxy]phenyl]-N1-(4-fluorophenyl)cyclopropane-1,1-dicarboxamide (foretinib); N-(4-((6,7-Dimethoxyquinolin-4-yl)oxy)phenyl)-N-(4-fluorophenyl)cyclopropane-1,1-dicarboxamide (cabozantinib(XL184)); N-(4-((6,7-Dimethoxyquinolin-4-yl)oxy)phenyl)-N-(4-fluorophenyl)cyclopropane-1,1-dicarboxamide (cabozantinib(XL184)); 3-[(1R)-1-(2,6-dichloro-3-fluorophenyl)ethoxy]-5-(1-piperidin-4-ylpyrazol-4-yl)pyridin-2-amine (crizotinib (Xalkori)); (3Z)—N-(3-Chlorophenyl)-3-({3,5-dimethyl-4-[(4-methylpiperazin-1-yl)carbonyl]-1H-pyrrol-2-yl}methylene)-N-methyl-2-oxo-2,3-dihydro-1H-indole-5-sulfonamide (SU11274); (3Z)-5-[[(2,6-Dichlorophenyl)methyl]sulfonyl]-3-[[3,5-dimethyl-4-[[(2R)-2-(1-pyrrolidinylmethyl)-1-pyrrolidinyl]carbonyl]-1H-pyrrol-2-yl]methylene]-1,3-dihydro-2H-indol-2-one hydrate (PHA-665752); 6-[[6-(1-Methylpyrazol-4-yl)-[1,2,4]triazolo[4,3-b]pyridazin-3-yl]sulfanyl]quinoline (SGX-523); 4-[1-(6-Quinolinylmethyl)-1H-1,2,3-triazolo[4,5-b]pyrazin-6-yl]-1H-pyrazole-1-ethanol methanesulfonate (1:1) (PF-04217903); 2-Fluoro-N-methyl-4-[7-[(quinolin-6-yl)methyl]imidazo[1,2-b]-[1,2,4]triazin-2-yl]benzamide (INCB28060); N-[4-[(3-Chloro-4-fluorophenyl)amino]-7-[[(3S)-tetrahydro-3-furanyl]oxy]-6-quinazolinyl]-4(dimethylamino)-2-butenamide (afatinib); 3-(5,6-Dihydro-4H-pyrrolo[3,2,1-ij]quinolin-1-yl)-4-(1H-indol-3-yl)-pyrrolidine-2,5-dione (ARQ-197 (Tivantinib)); N-[(2R)-1,4-dioxan-2-ylmethyl]-N-methyl-N-[3-(1-methyl-1H-pyrazol-4-yl)-5-oxo-5H-benzo[4,5]cyclohepta[1,2-b]pyridin-7-yl]sulfuric diamide (MK-2461); N-[4-(3-Amino-1H-indazol-4-yl)phenyl]-N′-(2-fluoro-5-methylphenyl)urea (Linifanib(ABT 869));

4-[[(3S)-3-Dimethylaminopyrrolidin-1-yl]methyl]-N-[4-methyl-3-[(4-pyrimidin-5-ylpyrimidin-2-yl)amino]phenyl]-3-(trifluoromethyl)benzamide (Bafetinib (INNO-406)); and pharmaceutical acceptable salts thereof.

In a further example, there is provided the pharmaceutical composition as described above, wherein the BCL family BH3-mimetic inhibitors include but are not limited to; 4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (ABT 263; Navitoclax);

2-[2-[(3,5-Dimethyl-1H-pyrrol-2-yl)methylene]-3-methoxy-2H-pyrrol-5-yl]-1H-indole methanesulfonate (Obatoclax mesylate (GX15-070)); 4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-Benzamide (ABT-737); pharmaceutically acceptable salts thereof.

In an additional example, there is provided the pharmaceutical composition as described above, wherein the ATM inhibitors comprise inhibitors include but are not limited: 2-Morpholin-4-yl-6-thianthren-1-yl-pyran-4-one (KU-55933); (2R,6S)-2,6-Dimethyl-N-[5-[6-(4-morpholinyl)-4-oxo-4H-pyran-2-yl]-9H-thioxanthen-2-yl]-4-morpholineacetamide (KU-60019); 1-(6,7-Dimethoxy-4-quinazolinyl)-3-(2-pyridinyl)-1H-1,2,4-triazol-5-amine (CP466722); α-Phenyl-N-[2,2,2-trichloro-1-[[[(4-fluoro-3-nitrophenyl)amino]thioxomethyl]amino]ethyl]benzene acetamide (CGK 733) and pharmaceutically acceptable salts thereof.

The present invention also provides the use of a peptide as described herein in the manufacture of a medicament for treating or preventing cancer. In some embodiments, the cancer as described above is characterized by overexpression or hyperactivity of eIF4E containing complexes. As used herein, the term “overexpression” denotes a level of expression of the proteins in a complex that comprises eIF4E that is above a level found in cells isolated or cultivated from a patient having no disease or being healthy. For example, overexpression of eIF4E may be found in the cancer cells isolated from a cancer patient as compared to the expression level of eIF4E in the non-cancer cells of the patient or in the cells isolated from an healthy patients, wherein the cells belong to the same group having the same histological, morphological, physical, and biological characteristics (e.g. hepatocytes, keratinocytes, lung cells . . . ). As used herein the term “hyperactivity” denotes a level of enzymatic, biological, dynamic or any measurable activity of the proteins in a complex that comprises eIF4E that is above a level found in cells isolated or cultivated from a healthy patient having no diseases, conditions or any ailments. For example, hyperactivity of eIF4E may be found in the cancer cells isolated from a cancer patient as compared to the activity level of eIF4E protein in the non-cancer cells of the patient or in the cells isolated from an healthy patients, wherein the cells belong to the same group having the same histological, morphological, physical, and biological characteristics. For example, the cells in which the expression or activity levels of eIF4E are compared may include but are not limited to hepatocytes, keratinocytes, or lung cells.

In some embodiments, the cancer treated or prevented in the invention may be any form of a cancer. Any forms of tumor or cancer may be used in the invention including for example, a benign tumor and a metastatic malignant tumor. Examples of cancers include, but are not limited to, gastric cancer, colon cancer, lung cancer, breast cancer, bladder cancer, neuroblastoma, melanoma, head and neck cancer, esophagus cancer, cervix cancer, prostate cancer and leukemia.

Other examples of tumors include, but are not limited to, haematological malignancies and solid tumours. Solid tumours include for instance a sarcoma, arising from connective or supporting tissues, a carcinoma, arising from the body's glandular cells and epithelial cells or a lymphoma, a cancer of lymphatic tissue, such as the lymph nodes, spleen, and thymus.

Thus, in some embodiments there is provided a method of treating or preventing cancer in a patient in need thereof. The method includes administering of a pharmaceutically effective amount of a peptide, the isolated nucleic acid or the vector as described above and herein. The method of the invention can in some embodiments include administering the pharmaceutically effective amount of the peptide with one or more further therapeutic compounds, wherein administration is simultaneous, sequential or separate.

The term “treat” or “treating” as used herein is intended to refer to providing an pharmaceutically effective amount of a peptide of the present invention or a respective pharmaceutical composition or medicament thereof, sufficient to act prophylactically to prevent the development of a weakened and/or unhealthy state; and/or providing a subject with a sufficient amount of the complex or pharmaceutical composition or medicament thereof so as to alleviate or eliminate a disease state and/or the symptoms of a disease state, and a weakened and/or unhealthy state.

In some embodiments, the cancer to be treated can include but is not limited to gastric cancer, colon cancer, lung cancer, breast cancer, bladder cancer, neuroblastoma, melanoma, head and neck cancer, esophagus cancer, cervix cancer, prostate cancer and leukemia.

In some embodiments, there is provided the use of a peptide as described above and herein for protein purification, or for inhibiting protein-protein interactions, or as template for protein-protein interactions.

In one example the peptide described herein is not or does not comprise the amino acid sequence KKRYSREFLLGF (SEQ ID NO: 1).

The term “pharmaceutically effective amount” as used herein means that amount of a modified eIF4E peptide as described above or a pharmaceutical composition or medicament comprising the peptide which is effective for producing some desired therapeutic effect in at least a sub-population of cells in the patient at a reasonable benefit/risk ratio applicable to any medical treatment.

Generally, an effective dosage per 24 hours may be in the range of about 0.0001 mg to about 1000 mg per kg body weight; suitably, about 0.001 mg to about 750 mg per kg body weight; about 0.01 mg to about 500 mg per kg body weight; about 0.1 mg to about 500 mg per kg body weight; about 0.1 mg to about 250 mg per kg body weight; or about 1.0 mg to about 250 mg per kg body weight. More suitably, an effective dosage per 24 hours may be in the range of about 1.0 mg to about 200 mg per kg body weight; about 1.0 mg to about 100 mg per kg body weight; about 1.0 mg to about 50 mg per kg body weight; about 1.0 mg to about 25 mg per kg body weight; about 5.0 mg to about 50 mg per kg body weight; about 5.0 mg to about 20 mg per kg body weight; or about 5.0 mg to about 15 mg per kg body weight.

A recent report has indicated that the small molecule ribavirin might interfere with the eIF4E:cap interaction and may therefore present a clinical opportunity as an eIF4E-targeted therapy. As anticipated, ribavirin treatment selectively diminished the expression of key, eIF4E-dependent proteins such as cyclin D1 and suppressed tumor growth. However, whether or not ribavirin actually binds eIF4E is controversial. Consequently, a rational methodology to develop small molecule inhibitors of the eIF4E: 7-methylguanosine cap interaction might be a fruitful approach for the development of an eIF4E-specific small molecule therapy. To date, no such drug-like inhibitors of the eIF4E-cap interaction have been reported.

An alternative approach to targeting the eIF4E-cap interaction is to selectively disrupt the interaction of eIF4E with eIF4G, thereby disabling the formation of the eIF4F complex. An alternative approach to targeting eIF4E would be to reduce eIF4E protein expression using antisense oligonucloetides (ASOs). eIF4E ASOs have been shown to effectively reduce both eIF4E RNA and protein in a wide array of transfected human and murine cells, subsequently reducing the expression of the malignancy-related proteins-specifically cyclin D1, VEGF, c-myc, survivin and BCL-2. Importantly, ASO mediated reduction of eIF4E did not affect the expression of β-actin, a protein encoded by a “strong” mRNA nor did it reduce overall protein synthesis substantially.

### Specific Illustrative Embodiments

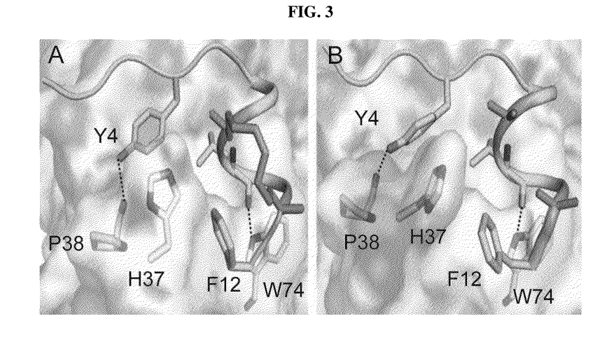

Inhibiting the protein interface between eIF4E and eIF4G is attractive for the design of anti-cancer therapeutics. Peptides derived from eIF4G1 and 4EBP1 (an inhibitor of the eIF4E:eIF4G interaction) that contain the residues responsible for their interactions with eIF4E (YXXXXLΦ motif, where Φ signifies any hydrophobic residue), are structural mimics of each other. The tyrosine (Y4) (FIG. 3) is engaged in multiple van der Waal contacts with eIF4E and an h-bond between its side chain hydroxyl and the carbonyl backbone of P38 of eIF4E. The leucine (L9) exploits a shallow cavity on the surface of eIF4E and interacts with W73 of eIF4E via an h-bond between its backbone and the indole of the tryptophan. The conserved hydrophobic residue (L10) packs against L131 and L135 of eIF4E. Crystal structures of both peptides complexed to eIF4E are approximately 50% α-helical; however they contain negligible helical content in solution.

Accordingly, the inventors build upon interactions between peptides and this interface and have ameliorated small molecules to inhibit the eIF4E and eIF4G interaction by rationally designing novel cross-linked peptide inhibitors to interrupt the eIF4E-eIF4G interface. As explained above and herein, cross-linking peptides entails the introduction of, for example, an all-hydrocarbon linkage between adjacent turns of the helix to stabilize the secondary structure of the peptide. This may enable: 1) improved affinity by reducing the entropic cost of binding, 2) prolonged in vivo half-life by increasing their proteolytic stability, 3) potential enhancement of their cellular uptake and intra-cellular activity.

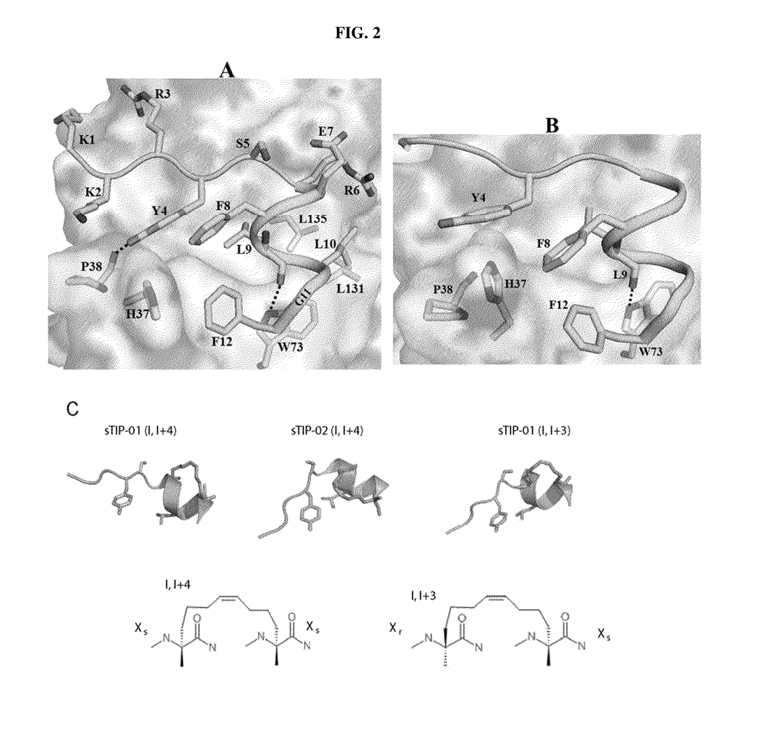

Hence there is provided the peptide as described herein, characterized in that the peptide is capable of inhibiting eIF4E and eIF4G interaction. The peptide sequence chosen as a template for design of stapled peptides against eIF4E was ¹KKRYSREFLLGF¹² (eIF4G^{D5S}) (SEQ ID NO: 1) or ¹KKRYSREFLLGFQF¹⁴ (SEQ ID NO: 16) and was derived from the eIF4G1 epitope that interacts with eIF4E. D5 was mutated to S to optimize the N-capping motif formed when the peptide is bound to eIF4E. The crystal structure of the eIF4G^{D5S} peptide bound to eIF4E was examined to identify sites for the insertion of a staple (FIG. 2A). Two i, i+4 staples were inserted at positions 7 and 11, and 8 and 12 respectively, to generate the stapled peptides sTIP-01 and 02. For sTIP-01, the two solvent exposed residues E7 and G11 were replaced in order to ensure that the peptide maintained its optimal interactions, whilst in sTIP-02, the hydrophobic staple was placed to replace the interactions made by F8 and F12 with eIF4E. Two i, i+7 staples were inserted at positions 7 and 14, 6 and 13, 5 and 12, and 8 and 15 respectively, to generate staples peptides sTIP05, sTIP06, sTIP07 and sTIP08 (see Table 1).

TABLE 1
Staple Attachment of TIP/sTIP Peptides

| | SEQ | |
|---|---|---|
| | ID NO | Sequence |
| eIF4G^{D5S} | 1 | KKRYSREFLLGF |
| TIP-01 | 2 | KKRYSRXFLLXF |
| TIP-02 | 3 | KKRYSREXLLGX |
| TIP-03 | 4 | KKRYSREXLLXF |
| TIP-04 | 5 | KKRYSRXQLLXL |
| TIP-01^{Tr} | 6 | KKRYSRXFLLX |
| TIP-01^{F12&} | 7 | KKRYSRXFLLX& |
| TIP-01^{F8A} | 8 | KKRYSRXALLXF |
| sTIP-01 | 9 | KKRYSR*FLL*F |
| sTIP-02 | 10 | KKRYSRE*LLG* |
| sTIP-03 | 11 | KKRYSRE*LL*F |
| sTIP-04 | 12 | KKRYSR*QLL*L |
| sTIP-01^{Tr} | 13 | KKRYSR*FLL* |
| sTIP-01^{F12&} | 14 | KKRYSR*FLL*& |
| sTIP-01^{F8A} | 15 | KKRYSR*ALL*F |
| Extended eIF4G1 epitope | 16 | KKRYSREFLLGFQF |
| sTIP-05 | 17 | KKRYSR*FLLGFQ* |
| sTIP-06 | 18 | KKRYS*EFLLGF*F |
| sTIP-07 | 19 | KKRY*REFLLG*QF |
| sTIP-08 | 20 | KKRYSRE*LLGFQF* |

X = AIB,
* = staple position,
& = 2AB = 2 amino butyric acid.

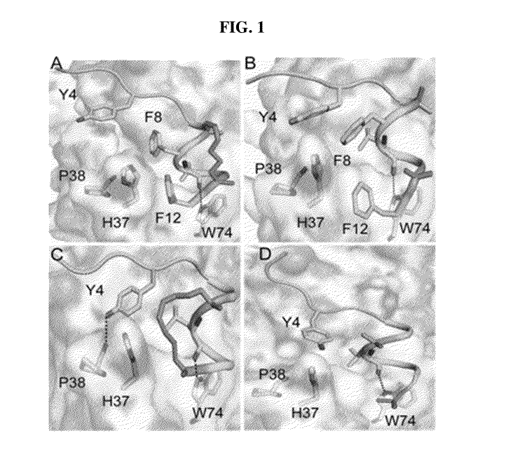

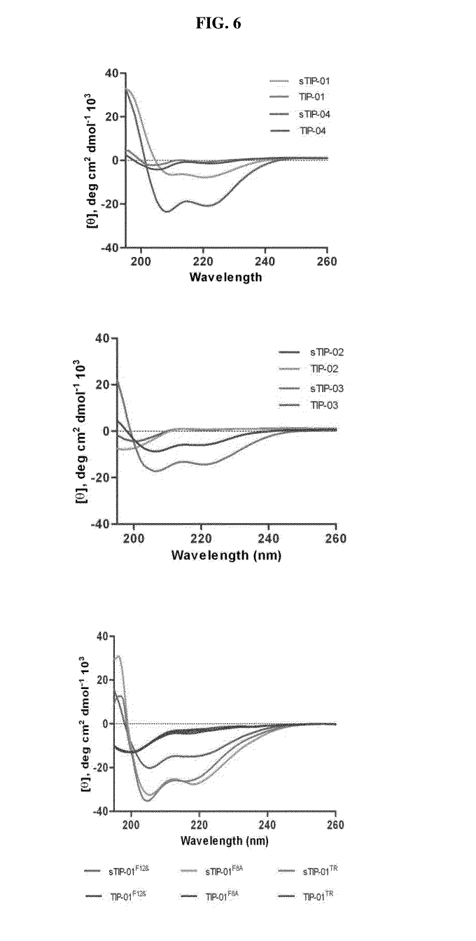

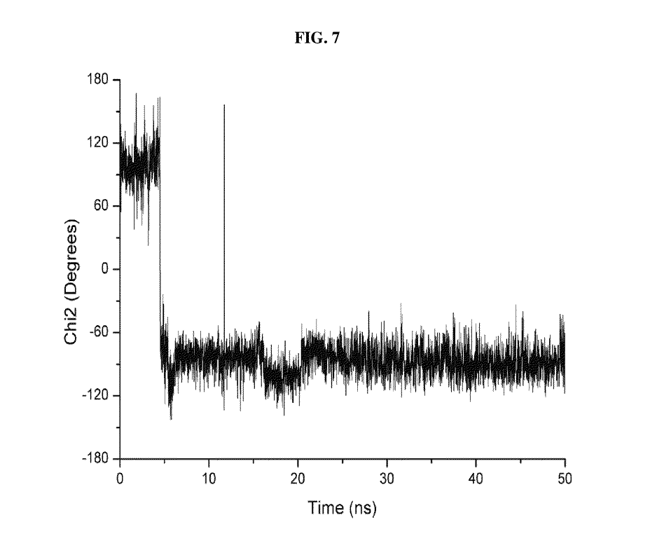

The K_{d} of sTIP-01 shows an ˜4.5 fold increase in K_{d} over the linear eIF4G^{D5S} peptide (Table 2). In contrast a diAIB derivative peptide, TIP-01, where the insertion sites of the staple have been replaced by aminoisobutyric acid (AIB), showed improved binding with an ˜2-fold decrease in the K_{d} over eIF4GD5S. CD spectra (FIG. 6 and Table 2) also revealed that the staple induces greater helicity in sTIP-01 than in TIP-01 or in eIF4G^{D5S}. Computer simulations reveal that the covalent staple in sTIP-01 imposes rigidity in the α-helix, increasing the strain on the network of interactions formed between H37, F8 and F12. This leads to steric occlusion of F8, causing a series of conformational changes to propagate along the peptide:protein interface. The sterically occluded F8 side-chain rotates around the χ-2 torsion angle and buries itself, quite favourably (Table 3), against the surface of eIF4E (FIGS. 7 and 1A). The F8 side chain now impedes Y4 from maintaining the conserved hydrogen bond with the backbone carbonyl of P38, causing Y4 to ‘flip out’ and become more exposed to the solvent, thereby reducing its energetic contribution to peptide:protein interactions (FIG. 1A, Tables 2 and 3). In TIP-01, the AIB substitutions impose less strain and there is enough flexibility in the helix for both F8 and F12 to interact with H37 in a binding mode that is similar, both structurally and energetically, to that seen in the eIF4GD5S crystal structure (FIGS. 1B and 2A; Table 3). The conserved h-bond between the indole of W73 and the backbone of L59 is highly stable in simulations for the sTIP-01, TIP-01 and eIF4GD5S complexes (Table 4). Interestingly, the other conserved h-bond (Y4:P38) is less stable even without the disruption imposed upon this interaction by the conformational changes in sTIP-01 (Table 2 and FIG. 2B).

sTIP-02 shows no improvement in binding over eIF4G^{D5S} (Table 2). However the AIB derivative of sTIP-02 (TIP-02) has a greatly reduced affinity for eIF4E indicating the importance of either F8 or F12 or both towards peptide:protein interaction. STIP-02 has less helicity than sTIP-01 in solution, but considerably more than TIP-02 (Table 2). Simulation of the sTIP-02:eIF4E complex shows that the staple interacts with the protein and contributes favourably to binding (FIG. 1C and Table 3). However, the lack in improvement of affinity suggests that the stapled peptide does not optimally replace the influence of F8 and F12. Further, in TIP-02, Y4 undergoes a conformational change and packs into the space previously occupied by F8 in the linear peptide and the staple in sTIP-02 (FIGS. 2A and 1D). This results in the loss of the Y4 hydrogen bond (Table 2) and weakens the interaction energy (Table 3), although the L9:W73 h-bond remains unaffected by these new interactions (Table 4).

An i, i+3 staple was used at positions 8 and 11 in the peptide (termed sTIP-03, Table 2) to mimic a helically stabilised eIF4G1 peptide. 10 The i, i+3 staple results in a 17-fold improvement in the K_{d}, unlike the I, I+4 staple in sTIP-01/02, in conjunction with a larger increase in its helicity (Table 2).

TABLE 2
Staple Attachment, Helicity, and
eIF4E Affinity of TIP/sTIP Peptides

| | | | | Helicity/ |
|---|---|---|---|---|
| | | | | Y4:P38 |
| | SEQ ID | | SPR/FP | Occupancy |
| | NO | Sequence | (K_{d},nM) | (%) |
| eIF4G^{D5S} | 1 | KKRYSREFLLGF | 99.9 ± 6.2/195.2 ± 12.1 | 0/10.4 |
| TIP-01 | 2 | KKRYSRXFLLXF | 59.7 ± 1.6/52.4 ± 6.4 | 2/49.6 |
| TIP-02 | 3 | KKRYSREXLLGX | 9189 ± 487/NA | 0/ |
| TIP-03 | 4 | KKRYSREXLLXF | 129.5 ± 1.0/288.8 ± 9.9 | 0/<5.0 |
| TIP-04 | 5 | KKRYSRXQLLXL | 22.0 ± 0.6/60.01 ± 3.3 | 4/95.8 |
| TIP-01^{Tr} | 6 | KKRYSRXFLLX | 12147 ± 2232/NA | 7/18.5 |
| TIP-01^{F12&} | 7 | KKRYSRXFLLX& | 110.7 ± 18.2/133.1 ± 5.6 | 11/86.2 |
| TIP-01^{F8A} | 8 | KKRYSRXALLXF | 117.3 ± 75.7/155.57 ± 4.5 | 10/86.2 |
| sTIP-01 | 9 | KKRYSR*FLL*F | NA/558.0 ± 59.5 | 24/22.8 |
| sTIP-02 | 10 | KKRYSRE*LLG* | 109.6 ± 4.6/146.7 ± 1.7 | 18/73.0 |
| sTIP-03 | 11 | KKRYSRE*LL*F | 3.4 ± 0.3/4.4 ± 0.6 | 45/90.3 |
| sTIP-04 | 12 | KKRYSR*QLL*L | 5.0 ± 0.7/11.5 ± 3.6 | 63/38.9 |
| sTIP-01^{Tr} | 13 | KKRYSR*FLL* | NA/NA | 72/29.2 |
| sTIP-01^{F12&} | 14 | KKRYSR*FLL*& | NA/159.8 ± 15.1 | 44/92.8 |
| sTIP-01^{F8A} | 15 | KKRYSR*ALL*F | NA/105.5 ± 6.3 | 80/5.13 |

X = AIB,
* = staple position,
& = 2AB = 2 amino butyric acid.

However the removal of F8 in TIP-03 only slightly attenuates binding with eIF4E indicating that F12 makes a larger contribution to the interaction, which is backed by simulations (Table 3). The h-bond between Y4 and P38 is also significantly stabilised (Table 2) and as a result increases the energetic contribution of Y4 to the interaction (Table 3). The L9:W73 h-bond remains highly favourable (Table 4). The restrained C-terminal F12 predominately packs against H37, which also forms van der Waals contacts with Y4 (FIG. 2A). The association of the conformationally more labile, diAIB analogue peptide (TIP-03) with eIF4E is characterized by an interaction network between F12, H37 and Y4 similar to that in sTIP-03 (FIG. 3B).

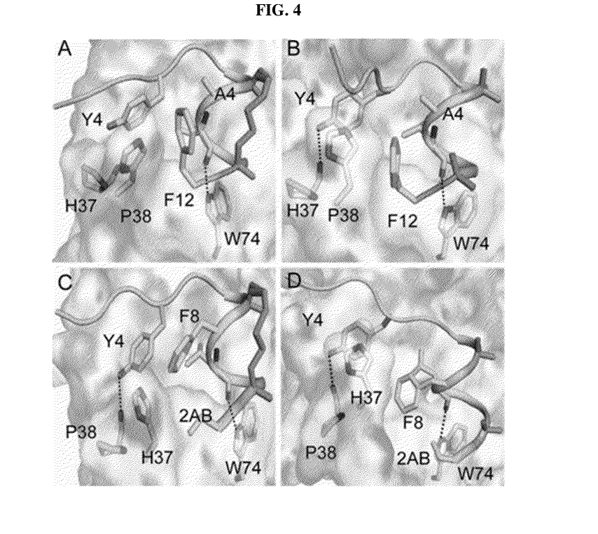

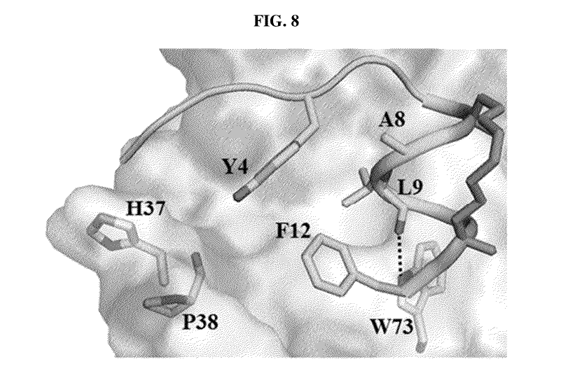

To optimise the packing of the peptide against eIF4E and alleviate the steric effects between F8 and F12 that occur in sTIP-01 (i, i+4), two point mutations (F^{8A} and F^{12&}, &=2-aminobutyric acid=2AB) were introduced into sTIP-01. Both these derivative peptides (sTIP-01F^{8A} and sTIP-01F^{12&}) showed improved K_{d} values as compared to sTIP-01 (Table 2). However the associated diAIB (TIP) substituted peptides still possessed higher, affinities for eIF4E reaffirming that the staple appears to limit the affinity. Both sTIP-01 derivatives have improved helicity compared to sTIP-01 with sTIP-01F^{8A} showing greater helical content than sTIP-01F12&. This suggests that F8 is more detrimental to helix stabilization than F12. Simulations of sTIP-01F^{8A} and TIP-01F^{8A} reveal that the interaction pattern between Y4, H37 and F12 influence the stability of the Y4:P38 h-bond. In sTIP-01F8A, H37 is found in two alternative states. The “out” conformation where F12 packs against Y4, which in turn occupies the space provided by F8A mutation (FIG. 8), or the “in” conformation where it forms a stacking interaction with Y4 and F12 (FIG. 4A). This conformational changes result in the rare formation of the h-bond (Table 2). In contrast, in TIP-01F8A, F12 shows no specific favourable interaction with H37 or Y4. Further, H37 interacts favourably with Y4 without disrupting its hydrogen bond interaction (FIG. 4B and Table 2). Comparatively, the Y4:P38 h-bond remains highly stable in simulations of the sTIP/TIP-01F^{12&} derivative peptides. In TIP-01F^{12&}, the C-terminal 2AB forms no interactions with H37.

Instead H37 forms hydrophobic interactions with Y4 and causes no disruption of the h-bond (FIG. 4D, Table 3). The incorporation of the i, i+4 staple induces a conformational change in the interactions formed by the peptide by restraining the C-terminal region of the helix. This causes 2AB to interact predominantly with H37 which in turn stacks with F8 resulting in a similar mode of binding as in eIF4G^{D5S} (FIGS. 4C and 6A, Table 3).

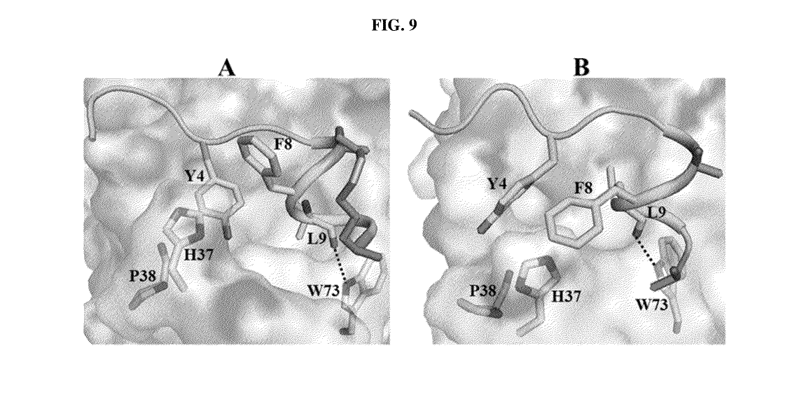

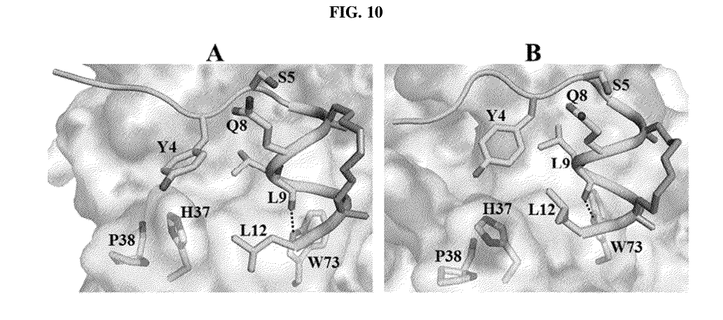

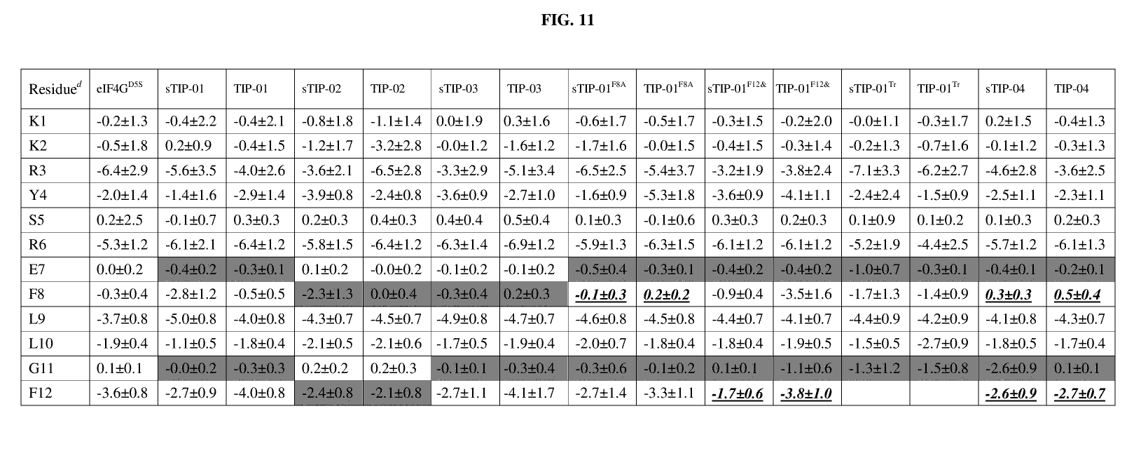

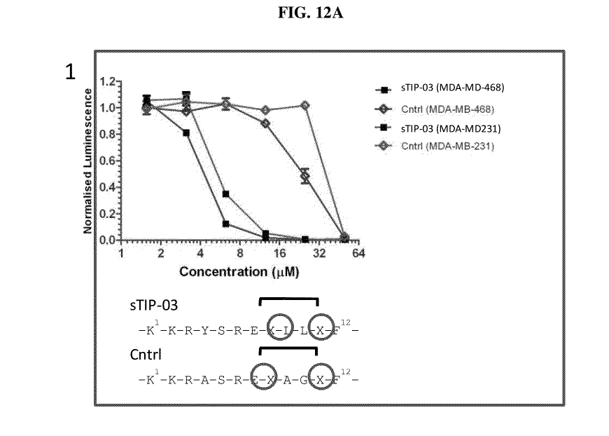

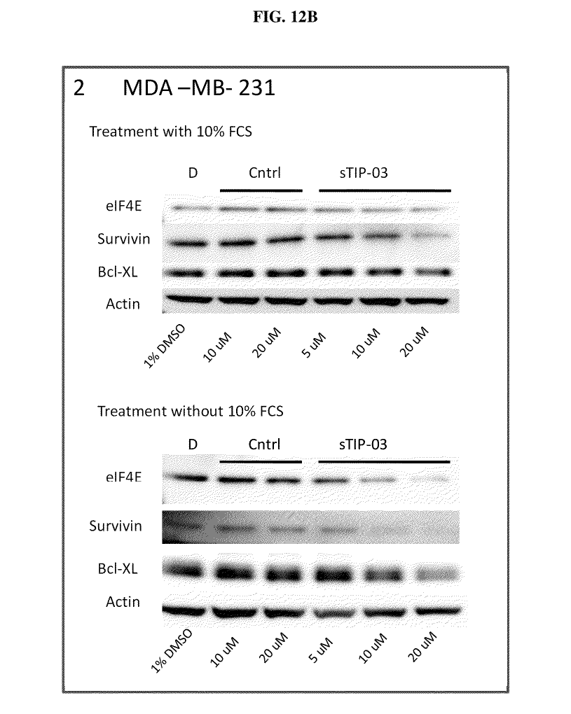

Removal of the C-terminal F12 residue of sTIP/TIP-01 which is shown to contribute significantly to binding (Table 3) results in a peptide with negligible affinity for eIF4E (Table 2), further emphasizing the importance of this residue. Simulations of both peptide derivatives (Termed sTIP-01^{Tr} and TIP-01^{Tr}) showed dramatic rearrangements of the side-chain packing interactions of Y4, F8 and H37 (FIGS. 9A and 9B). These rearrangements are principally driven by the lack of an interaction at the C-terminal of the peptide.

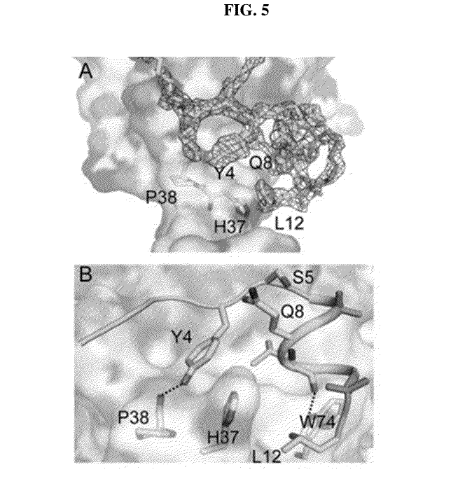

The following mutations (F8Q and F12L) previously identified by phage display were introduced into an i, i+4 stapled peptide termed sTIP-04. The resulting peptide had a significant increase in helicity and a 25 fold improvement in K_{d} compared to eIF4G^{D5S} (Table 2). Crystallization of the sTIP-04:eIF4E complex (FIG. 5C) confirmed the results of a previous study that the S5 side-chain forms an interaction network with the Q8 side-chain and the backbone amides on the first turn of the peptide helix, thus stabilizing the bound complex. Simulations showed that the L9:W73 hydrogen bond in both derivative peptides (sTIP-04 and TIP-04) is very stable (Table 4). However the Y4:P38 hydrogen bond is attenuated in the presence of the staple (Table 2) compared to the AIB derivative. In TIP-04 the optimal packing of H37, L12 and Y4 does not disrupt the conserved hydrogen bond (FIG. 5A). In the stapled derivative, H37 forms more favourable van der waals contacts with L12, as a result of the staple rigidifying the C-terminal, which causes Y4 to undergo a transition in order to maintain favourable packing (FIG. 5B). It is this favourable packing rearrangement as can be seen from the energetic contribution of Y4 (Table 3) that causes the attenuation of the Y4:P38 h-bond. Simulation starting from sTIP-04:eIF4E crystal structure also showed similar behaviour as was observed for sTIP-04 derivative system (FIG. 5D).

TABLE 4
Hydrogen bond occupancy and solvent accessibility from the
respective simulations.

| | L9:W73 | Y4 |
|---|---|---|
| Peptide | (% Occupancy) ^{a} | SASA (Å²)^{b} |
| eIF4G^{D5S} (SEQ ID NO: 1) | 91.3 | 58.7 ± 27.9 |
| sTIP-01 (SEQ ID NO: 9) | 98.6 | 84.7 ± 45.0 |
| TIP-01 (SEQ ID NO: 2) | 97.4 | 34.2 ± 20.0 |
| sTIP-02 (SEQ ID NO: 10) | 99.0 | 21.1 ± 8.8 |
| TIP-02 (SEQ ID NO: 3) | 96.3 | 72.8 ± 19.2 |
| sTIP-03 (SEQ ID NO: 11) | 99.4 | 30.2 ± 13.3 |
| TIP-03 (SEQ ID NO: 4) | 97.9 | 40.8 ± 15.0 |
| sTIP-01^{F8A} (SEQ ID NO: 15) | 97.3 | 40.7 ± 19.1 |
| TIP-01^{F8A} (SEQ ID NO: 8) | 99.1 | 21.4 ± 10.9 |
| sTIP-01^{F12&} (SEQ ID NO: 14) | 99.5 | 22.0 ± 9.8 |
| TIP-01^{F12&} (SEQ ID NO: 7) | 98.5 | 25.0 ± 11.7 |
| sTIP-01^{Tr} (SEQ ID NO: 13) | 94.3 | 56.7 ± 38.6 |
| TIP-01^{Tr} (SEQ ID NO: 6) | 96.4 | 52.0 ± 21.0 |
| sTIP-04 (SEQ ID NO: 12) | 97.9 | 35.7 ± 22.7 |
| TIP-04 (SEQ ID NO: 5) | 95.8 | 23.2 ± 11.0 |

^{a} Percentage of time the hydrogen bond criteria (distance and angle cut-off of 3.5 Å and 120° respectively) was satisfied between the backbone oxygen of L9 in the peptide and the indole nitrogen of W73 in eIF4E analyzed in a total of 5000 structures per simulation.
^{b}Solvent accessible surface area (SASA) of Y4 residue in the peptide.

The design of high affinity binding eIF4E stapled peptides highlight that restraining the conformational freedom of peptides via hydrocarbon linkages can have very dramatic effects upon the structural dynamics and avidity of the peptide:protein interface. This is aptly shown by sTIP-01 where F8 undergoes a conformational constraint arising from steric occlusions caused by over stabilization of the helix. The effects on packing at the protein:peptide interface can be more subtle. Both sTIP-03 (I, I+3) and sTIP-01^{F8A}. (I, I+4) lack F8, but the position of the staple influences the precise interaction of F12 with H37. The different staples result in changes in the conformational space accessible to F12 and this in turn affects the stability of the conserved Y4:P38 h-bond. For sTIP-01^{F8A} the Y4 packs into the space created as a result, of the large-to-small sidechain mutation in F8A, resulting in the loss of the Y4:P38 h-bond. In contrast, in sTIP-03, F12 interacts with H37 and prevents Y4 forming extra hydrophobic contacts, thus resulting in the preservation of the h-bond. The inventors also found that alternative helical stabilization strategies give rise to diverse molecular mechanisms for binding and that improvements in affinity result from compensatory interactions. Staples predominately increase the helicity of the peptide in solution before binding but this can be compromised by non-optimal interactions at the peptide:protein interface. In the two high affinity peptides designed (sTIP-04 and sTIP-03) such limitations have been overcome by optimising packing effects at the interface, stabilising the bound complex and greater helical stabilization in solution. With sTIP-03, the staple only induces 45% helicity but this is compensated for with the formation of the Y4:P38 h-bond and by optimal packing interactions of F12. In contrast, sTIP-04 loses the Y4:P38 hydrogen bond upon binding but compensation arises via greater helicity (63%) in solution and stabilisation of the helical bound form by Q8.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

Materials and Methods

Linear peptides (TIP) were ordered from and synthesized by Mimotopes, Clayton, Australia. Stapled peptides (sTIP) were synthesized by Anaspec (San Diego, Calif.). Stapled peptides (sTIP) with an (i, i+4) hydrocarbon linkage were generated by replacing the respective amino acids with the olefin-bearing unnatural amino acids (5)-2-(4′-pentenyl) alanine and (S)-2-(4′-pentenyl) alanine and stapled via olefin metathesis using the Grubbs catalyst. Stapled peptides (sTIP) with an (i, i+3) hydrocarbon linkage were generated by replacing the respective amino acids with the olefin-bearing unnatural amino acids (R)-2-(4′pentenyl) alanine and (S)-2-(4′-pentenyl) alanine and stapled via olefin metathesis using the Grubbs catalyst. The stapled peptides were purified using HPLC to >90% purity. All peptides were amidated at their C-terminus and acetylated at their N-terminus. The linear peptide with the C-terminal FAM labeled lysine (KKRYSRDFLLALQK-(FAM)) was synthesized by Mimotopes (Clayton, Australia) with the N-terminal acetylated and was purified using HPLC to >90% purity.

Protein Expression and Purification

Full-length human eIF4E was expressed and purified as described in Brown, C. J., et al., (2007), Crystallographic and mass spectrometric characterisation of eIF4E with N7-alkylated cap derivatives. J Mol Biol 372, 7-15.

Circular Dichroism Studies

CD was measured on a JASCO J-810 spectropolarimeter, and spectra were either recorded with a quartz cuvette (Helmer) with a pathlength of 0.01 cm or 0.1 cm in 5 mM sodium phosphate buffer (pH 7.0). FUV CD spectra were recorded from 260 nm to 200 nm at a peptide concentration of either 2 mg/ml or 0.5 mg/ml, respectively. The CD signal was converted into mean residue ellipticity in units of degree·cm²·dmol⁻¹·residue⁻¹. CD spectra were recorded at a data pitch of 0.2 nm at 50 nm min⁻¹, a response time of 2 s, and a bandwidth of 2 nM. Once converted to mean residue ellipticity, percent α-helicity can be calculated with equation (1) and (2):

$$\begin{matrix}\% Helicity = 100 \times ( \frac{{[ θ ]}_{222}}{{[ θ ]}_{222}^{ma x}} ) \\ Where & ( 1 ) \\ {[ θ ]}_{222}^{ma x} = - 40 \text{,} 000 \times [ 1 - ( \frac{2.5}{x} ) ] \\ x = number of amino acids & ( 2 )\end{matrix}$$

Fluorescence Anisotropy Assays and K_{d} Determination

Purified full length eIF4E was titrated against 50 nM carboxyfluorescein (FAM) labeled tracer peptide (KKRYSRDFLLALQK-(FAM)). The dissociation constants for the titration of eIF4E against the FAM labeled tracer peptide were determined by fitting the experimental data to a 1:1 binding model equation shown below:

$$r = {r}_{o} + ( {r}_{b} - {r}_{o} ) \times \frac{( {K}_{d} + {[ L ]}_{t} + {[ P ]}_{t} ) - \sqrt{{{K}_{d} + {[ L ]}_{t} + {[ P ]}_{t} )}^{2} - {{4 [ L ]}_{t} [ P ]}_{t}}}{{2 [ L ]}_{t}}$$

where [P] is the protein concentration (eIF4E), [L] is the labeled peptide concentration, r is the anisotropy measured, r0 is the anisotropy of the free peptide, r_{b} is the anisotropy of the eIF4E-FAM-labeled peptide complex, K_{d} is the dissociation constant, [L]ₜ is the total FAM labeled peptide concentration, and [P]ₜ is the total eIF4E concentration. The determined apparent K_{d} value (shown in the table below) were used in determining the apparent K_{d} values in subsequent competition assays for the respective competing ligands:

| Peptide | eIF4E K_{d} |
|---|---|
| Ac-KKRYSRDFLLALQK-(FAM) | 50.3 nM |

Apparent K_{d} values were determined for a variety of molecules via competitive anisotropy anisotropy experiments. Titrations were carried out with the concentrations eIF4E held constant at 200 nM, respectively and the labeled peptide at 50 nM. The competing molecules were then titrated against complex of the FAM labeled peptide and protein. Apparent K_{d} values were determined by fitting the experimental data to the equations shown below:

$$r = {r}_{o} + ( {r}_{b} + {r}_{o} ) \times \frac{2 \sqrt{( {d}^{2} - 3 e )} cos ( θ / 3 ) - 9}{3 {K}_{d 1} + 2 \sqrt{( {d}^{2} - 3 e )} cos ( θ / 3 ) - d}$$

$$d = {K}_{d 1} + {K}_{d 2} + {[ L ]}_{st} + {[ L ]}_{t} - {[ P ]}_{t}$$

$$e = ( {[ L ]}_{t} - {[ P ]}_{t} ) {K}_{d 1} + ( {[ L ]}_{st} - {[ P ]}_{t} ) {K}_{d 2} + {K}_{d 1} {K}_{d 2}$$

$$f = - {K}_{d 1} {{K}_{d 2} [ P ]}_{t}$$

$$θ = ar cos [ \frac{- 2 {d}^{3} + 9 de - 27 f}{2 \sqrt{{( {d}^{2} - 3 e )}^{3}}} ]$$

[L]ₛₜ and [L]ₜ denote labeled ligand and total unlabeled ligand input concentrations, respectively. K_{d2} is the dissociation constant of the interaction between the unlabelled ligand and the protein. In all competitive types of experiments, it is assumed that [P]ₜ>[L]ₛₜ, otherwise considerable amounts of free labeled ligand would always be present and would interfere with measurements. K_{d1} is the apparent K_{d} for the labeled peptide used in the respective experiment, which has been experimentally determined as described in the previous paragraph. The FAM-labeled peptide were dissolved in DMSO at 1 mM and diluted into experimental buffer. Readings were carried out with an Envision Multilabel Reader (PerkinElmer). Experiments were carried out in PBS (2.7 mM KCl, 137 mM NaCl, 10 mM Na₂HPO₄ and 2 mM KH₂PO₄ (pH 7.4)) and 0.1% Tween 20 buffer. All titrations were carried out in triplicate. Curve-fitting was carried out using Prism 4.0 (GraphPad).

To validate the fitting of a 1:1 binding model we carefully analyzed that the anisotropy, value at the beginning of the direct titrations between eIF4E and the FAM labeled peptide did not differ significantly from the anisotropy value observed for the free fluorescently labeled peptide. Negative control titrations of the ligands under investigation were also carried out with the fluorescently labeled peptide (in the absence of eIF4E) to ensure no interactions were occurring between the ligands and FAM labeled peptide. In addition it was ensured that the final baseline in the competitive titrations did not fall below the anisotropy value for the free fluorescently labeled peptide, which would otherwise indicate an unintended interaction between the ligand and the FAM labeled peptide to be displaced from the eIF4E binding site K_{d}s were not calculated for TIP-02 and TIP-01^{Tr} as they failed to displace the FAM labeled peptide in the competition-assay.

Surface Plasmon Resonance

For stock peptide solutions, the compounds were dissolved in 100% DMSO to a concentration of 10 mM; further dilutions of the peptide stock solutions into DMSO and/or running buffer were performed immediately prior to analysis. Running buffer consisted of 10 mM Hepes pH 7.6, 0.15M NaCl, 1 mM DTT and 0.1% Tween20. Stock/DMSO diluted peptide solutions were diluted into 1.03× running buffer to make a peptide solution with 3% DMSO final concentration. Working concentrations of peptide were reached with further dilution of samples into running buffer which contained 3% DMSO. Surface Plasmon resonance experiments were performed on a Biacore T100 machine.

eIF4E was immobilized on a CM5 sensor chip. The CM5 chip was conditioned with a 6 s injection of 100 mM HCL, followed by a 6 s injection of 0.1% SDS and completed with a 6 s injection of 50 mM NaOH at a flow rate of 100 μl/min. Activation of the sensor chip surface was performed with a 1:1 mixture of NHS (115 mg ml⁻¹) and EDC (750 mg ml⁻¹) for 7 min at 10 μl min⁻¹. Purified eIF4E was diluted with 10 mM sodium acetate buffer (pH 5.0) to a final concentration of 0.5 μM with m⁷ GTP present in a 2:1 ratio in order to stabilize eIF4E. The amount of eIF4E immobilized on the activated surface was controlled by altering the contact time of the protein solution and was approximately 1000 RU. After the immobilization of the protein, a 7-min injection (at 10 μl min⁻¹) of 1 M ethanolamine (pH 8.5) was used to quench excess active succinimide ester groups.

Six buffer blanks were first injected to equilibrate the instrument fully and then a solvent correction curve was performed followed by a further two blank injections. The solvent correction curve was setup by adding varying amount of 100% DMSO to 1.03× running buffer to generate a range of DMSO solutions (3.8%, 3.6%, 3.4%, 3.2%, 3%, 2.85%, 2.7% and 2.5% respectively). Using a flow rate of 50 μl/min, compounds were injected for 60 s and dissociation was monitored for 180 s. The data collection rate was 10 Hz. K_{d}s were determined using the BiaEvaluation software (Biacore) and calculated kinetically from the dissociation and association phase data for each of the peptides. The kinetic data were fitted to 1:1 binding models. Each individual peptide K_{d} was determined from three separate titrations. Within each titration at least two concentration points were duplicated to ensure stability and robustness of the chip surface. K_{d}s were not calculated for sTIP-01, sTIP-01^{Tr}, sTIP-01^{F12&} and sTIP-01^{F8A} variant stapled peptides as the sensograms exhibited anomalous sensograms with non-stoichiometric binding under the conditions tested.

Computer Simulations

Starting Structures

Crystal structure of eIF4G^{D5S} peptide in complex with eIF4E protein¹ solved at a resolution of 2.16 Å was downloaded from the Protein Data Bank (PDB ID: 4AZA). Only Chain A and B were taken for the current study. The unresolved residues in the structure from S209 to T211 were modeled using eIF4G1:eIF4E² (PDB ID: 2W97) structure as a template. Residue range from P31-V217 numbered according to its Uniprot ID P06730 is considered for eIF4E protein in this study. The sequence of the twelve residue peptide (eIF4G^{D5S}) is “KKRYSERFLLGF” and is sequentially numbered from 1-12 in the main text. The hydrocarbon linker in the various designed stapled peptides for this work was modeled using the XLEAP module of AMBER. RESP (Restrained ElectroStatic Potential) based atomic charges for the hydrocarbon linker were derived using the R.E.D. server interface by employing the RESP-A1A (HF/6-31G*) charge model and Guassian_{—}2009_C.01 quantum mechanics program. Other force field parameters for the linker were derived from all-atom ff99SB force field in AMBER11. Modified amino acids used in this study such as AIB (Amino isobutryric acid) and 2AB (2 Amino butyric acid) were modeled and their force field parameters were subsequently derived in a similar manner. In-silico mutations on the peptide were performed using PyMOL (Schrodinger) molecular visualization software. The N-terminal of the protein and peptide was acetylated (ACE) while the C-terminal was methylated (NME) for the protein and amidated (NHE) in the case of the peptide. This was done in accordance to what was followed for experimental binding studies (See Table 2 of main text). The starting structures were placed in a cuboid water box such that the minimum distance from the edge of the box was, at-least 12 Å. TIP3P water model was used for solvation. The solvated systems were neutralized by adding appropriate numbers of chloride ions using the TLEAP module of AMBER. Total of fifteen systems were prepared (See Table 2 of the main text). An additional simulation of sTIP-04: eIF4E system was also performed starting from the solved crystal structure (PDB ID:XXXX) as mentioned in the main text. The total number of atoms in the system ranged from 37,401 to 38,199. [S&F: Dear Inventors, please let us know the PDB ID of the crystal structure]

Simulations

Molecular Dynamics Simulations were performed using the PMEMD module of AMBER11 employing the all-atom ff99SB force field parameters. The solvated systems were initially relieved of any unfavourable interactions by subjecting them to 1000 steps of energy minimization which included using 500 steps each of steepest descent followed by conjugate gradient algorithms. This was performed in three steps. The first involved imposing Cartesian restrain on the solute while the solvent molecules were allowed to relax around it. This was followed by restraining the solvent while the solute was energy minimized. The final stage was done with no restrain and the whole system was energy minimized. Force constant of 500 kcal mol⁻¹ Å² was used during the restraining steps. The systems were then gradually heated to 300K over a period of 30 ps using the NVT ensemble. Following this, each system was equilibrated under NPT conditions for 500 ps. They were then subjected to the production phase of molecular dynamics simulation using the NPT ensemble for a simulation period of 50 ns each. Total of sixteen all-atom molecular dynamics simulations were performed in explicit solvent which amounts to a total of 800 ns of simulation time. Simulation temperature of 300K was maintained using langevin dynamics with collision frequency of 1.0 ps⁻¹ and the pressure was maintained at 1 atm using weak-coupling with pressure relaxation time of fps. Periodic boundary conditions in x, y and z directions was appropriately applied. Particle Mesh Ewald method (PME) was used for treating the long range electrostatic interactions. All bonds involving hydrogen atoms were constrained using the SHAKE algorithm. A time step of 2fs was used and the coordinates were saved every 1 ps. All representative figures shown in this work were generated using PyMOL (Schrodinger).

Energy Decomposition and Hydrogen Bond

Molecular free energy decomposition based on the MM/GBSA (Molecular Mechanics/Generalized Born Surface Area) analysis was performed on the simulated trajectories in order to obtain a quantitative description of the energetic contribution for the peptide:protein interaction. This was done using the MMPBSA.py script in AMBER. For the analysis, 1000 structures were extracted from the 50 ns production phase of molecular dynamics simulation at an interval of every 50 ps. Water molecules and CF ions were stripped from the extracted structures and the solvent effect was incorporated using a Generalized Born Solvation Model. Salt concentration of 0.15 mM was used. The surface area was calculated by employing a recursive method (ICOSA) which approximates a sphere around an atom beginning from an icosahedra shape. The hydrogen bond analysis was performed using the PTRAJ module in AMBER using a distance and angle cut-off of 3.5 Å and 120° respectively for structures extracted at every 10 ps from the production phase of simulated trajectories.

Crystallization

The eIF4E:eIF4G1-sTIP-04 (stapled peptide) complex was crystallized by vapor diffusion using the sitting drop method. Crystallization drops were setup with eIF4E and sTIP-04 (stapled peptide) at concentrations of 150 μM and 450 μM respectively. Sitting drops were set up in 48 well Intelli-Plates (Hampton research) with 1 μl of the protein sample mixed with 1 μl of the mother-well solution. Crystals grew over a period of one week in 18-26% of Polyethylene glycol 3350, 0.01-02M Ammonium Sulphate and 100 mM Bis-Tris at pH 5.5. For X-ray data collection at 100 K, crystals were transferred to an equivalent mother liquor solution containing 20% (v/v) glycerol and then flash frozen in liquid nitrogen.

Data Collection and Refinement

The data was collected on a X8 Proteum rotating anode source (Bruker) using a CCD detector. The crystal diffracted to a resolution of 2.6 Å and was integrated and scaled using PROTEUM2 (Bruker). The initial phases of the binary complexed crystals of eIF4E were solved by molecular replacement with the program PHASER using the human eIF4E structure complexed with the eIF4G1 peptide (PDB accession code: 2W97) as a search model. The starting models were subjected to rigid body refinement and followed by iterative cycles of manual model building in Coot and restrained refinement with TLS in Refmac 6.0. Models were validated using PROCHECK and the MOLPROBITY webserver. Final models were analysed using PYMOL (Schrödinger). See table 4 for data collection and refinement statistics. The eIF4E complex structure has been deposited in the PDB under the submission code 4BEA.PDB.

1. An isolated peptide comprising or consisting of the amino acid sequence of:
(SEQ ID NO: 21)
K¹K²R³Y⁴Xaa₁Xaa₂Xaa₃Xaa₄L⁹L¹⁰Xaa₅Xaa₆Xaa₇Xaa₈Xaa₉
wherein:
Xaa₁ is selected from the group consisting of S (serine), aminoisobutyric acid and an unnatural amino acid;
Xaa₂ is selected from the group consisting of R (arginine), aminoisobutyric acid and an unnatural amino acid;
Xaa₃ is selected from the group consisting of E (glutamic acid), aminoisobutyric acid and an unnatural amino acid;
Xaa₄ is selected from the group consisting of F (phenylalanine), Q (glutamine), A (alanine), aminoisobutyric acid and an unnatural amino acid;
Xaa₅ is selected from the group consisting of G (glycine), aminoisobutyric acid and an unnatural amino acid;
Xaa₆ is selected from the group consisting of F (phenylalanine), L (leucine), aminoisobutyric acid, 2-aminobutyric acid and an unnatural amino acid;
Xaa₇ is absent or selected from the group consisting of Q (glutamine), aminoisobutyric acid and an unnatural amino acid;
Xaa₈ is absent or selected from, the group consisting of F (phenylalanine), aminoisobutyric acid and an unnatural amino acid;
Xaa₉ is absent or selected from the group consisting of aminoisobutyric acid and an unnatural amino acid;
wherein the peptide comprises at least one peptide-cross linker linking Xaa₁, Xaa₂, Xaa₃ or Xaa₄ with Xaa₅, Xaa₆, Xaa₇, Xaa₈ or Xaa₉.

2. The peptide of claim 1 comprising at least two peptide cross linkers.

3. The peptide of claim 2, wherein the unnatural amino acid is in the position Xaa₁, Xaa₂, Xaa₃ or Xaa₄ and cross-links to position Xaa₅, Xaa₆, Xaa₇, Xaa₈ or Xaa₉.

4. The peptide of any one of the preceding claims wherein the cross-linker is a hydrocarbon linkage.

5. The peptide of any one of the preceding claims, wherein the unnatural amino acid at position Xaa₁, Xaa₂, Xaa₃ or Xaa₄ that cross-links the unnatural amino acid to position Xaa₅, Xaa₆, Xaa₇, Xaa₈ or Xaa₉ are both olefin-bearing unnatural amino acids.

6. The peptide of claim 5, wherein the olefin-bearing unnatural amino acid is selected from the group consisting of (S)-2-(4′-pentenyl)alanine, (R)-2-(4′-pentenyl)alanine, (S)-2-(7′-octenyl)alanine, (R)-2-(7′-octenyl)alanine and any one of the aforementioned amino acids with varied length.

7. The peptide of any one of claims 1 to 3, wherein the cross-linker is a cysteine bridge or a Lys-Asn (Lysine-Asparagine) linker.

8. The peptide of any one of the preceding claims, wherein the C-terminus of the peptide is amidated.

9. The peptide of any one of the preceding claims, wherein the N-terminus of the peptide is acetylated.

10. The peptide of any one of the preceding claims, characterized in that the peptide is capable of inhibiting eIF4E and eIF4G interaction.

11. The peptide of any one of the preceding claims, wherein the peptide is modified to include one or more ligands selected from the group consisting of: hydroxyl, phosphate, amine, amide, sulphate, sulphide, a biotin moiety, a carbohydrate moiety, a fatty acid-derived acid group, a fluorescent moiety, a chromophore moiety, a radioisotope, a PEG linker, an affinity label, a targeting moiety, an antibody, a cell penetrating peptide and a combination of the aforementioned ligands.

12. The peptide of any one of the preceding claims, wherein the peptide comprises formula I: 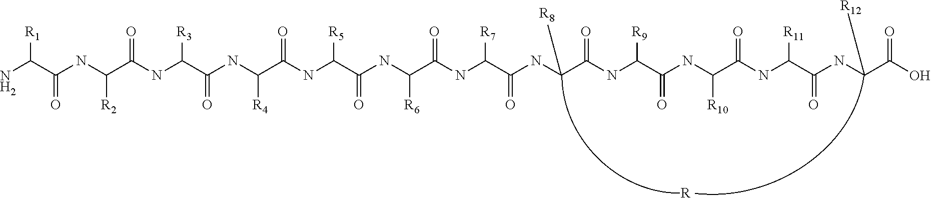
wherein:
R₁ and R₂ are —(CH₂)₄—NH₂ [K];
R₃ is —(CH₂)₃—NH—C(NH₂)₂ [R];
R₄ is —CH₂-Phenyl-OH [Y];
R₅ is —CH₂—OH [S];
R₆ is —(CH₂)₃—NH—C(NH₂)₂ [R];
R₇ is —(CH₂)₂C(O)OH [E], or aminoisobutyric acid;
R₈ and R₁₂ are independently H, a C₁ to C₁₀ alkyl, alkenyl, alkynyl, arylalkyl, cycloalkylalkyl, heteroarylalkyl, or heterocyclylalkyl;
R₉ and R₁₀ are —CH₂CH(CH₃)₂ [L]; and
R₁₁ is —H [G] or aminoisobutyric acid;
and wherein
R is anyone of alkyl, alkenyl, alkynyl, or [R′—K—R″]n; each of which is substituted with 0-6 R₁₃;
R′ and R″ are independently alkylene, alkenylene or alkynylene;
each R₁₃ is independently halo, alkyl, OR₁₄, N(R₁₄)₂, SR₁₄, SOR₁₄, SO₂R₁₄, CO₂R₁₄, R₁₄, a fluorescent moiety, or a radioisotope;
K is independently O, S, SO, SO₂, CO, CO₂, or CONR₁₄;
each R₁₄ is independently H, alkyl, or a therapeutic agent;
n is an integer from 1-4.

13. The peptide of claim 12, wherein R₈ and R₁₂ are independently H or a C₁ to C₆ alkyl.

14. The peptide of claim 13, wherein R₈ and R₁₂ are each —CH₃ [A] and R is a cyclooctenyl (sTIP-02) cross-linking the α-carbon of the two unnatural amino-acids.

15. The peptide of claim 14, wherein R is obtained by cross-linking the pentenyl side chains having the S stereochemistry of (S)-2-(4′-pentenyl)alanine at position Xaa₂ (i; R₈ is CH₃) and at position Xaa₄ at a position four amino acids apart (i+4; R₁₂ is CH₃), wherein the pentenyl side chains of both Xaa₂ and Xaa₄ are linked to the α-carbon of the two unnatural amino-acids and are on the same side of the α-helix (sTIP-02).

16. The peptide of any one of claims 1 to 11, wherein the peptide comprises formula II: 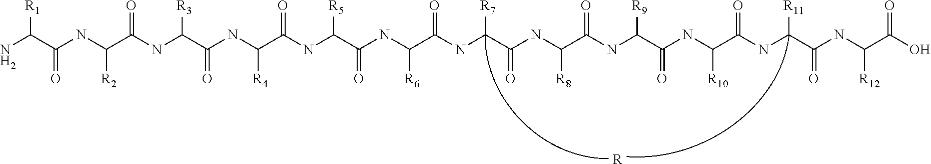
wherein:
R₁ and R₂ are —(CH₂)₄—NH₂ [K];
R₃ is —(CH₂)₃—NH—C(NH₂)₂ [R];
R₄ is —CH₂-Phenyl-OH [Y];
R₅ is —CH₂—OH [S];
R₆ is —(CH₂)₃—NH—C(NH₂)₂ [R];
R₇ and R₁₁ are independently H, a C₁ to C₁₀ alkyl, alkenyl, alkynyl, arylalkyl, cycloalkylalkyl, heteroarylalkyl, or heterocyclylalkyl;
R₈ is benzyl [F], or —(CH₂)₂—C(O)NH₂ [Q], or —CH₃ [A], or aminoisobutyric acid;
R₉ and R₁₀ are —CH₂CH(CH₃)₂ [L]; and
R₁₂ is benzyl [F], or —CH₂CH(CH₃)₂ [L], or aminoisobutyric acid or 2-aminobutyric acid;
and wherein
R is anyone of alkyl, alkenyl, alkynyl, or [R′—K—R″]n; each of which is substituted with 0-6 R₁₃;
R′ and R″ are independently alkylene, alkenylene or alkynylene;
each R₁₃ is independently halo, alkyl, OR₁₄, N(R₁₄)₂, SR₁₄, SOR₁₄, SO₂R₁₄, CO2R14, R₁₄, a fluorescent moiety, or a radioisotope;
K is independently O, S, SO, SO₂, CO, CO₂, or CONR₁₄;
each R₁₄ is independently H, alkyl, or a therapeutic agent;
n is an integer from 1-4.

17. The peptide of claim 16, wherein R₇ and R₁₁ are independently H or a C₁ to C₆ alkyl.

18. The peptide of claim 16, wherein R₇ and R₁₁ are each CH₃ (methyl) and;
wherein R is 4′-cyclooctenyl and;
wherein R₈ is benzyl [F] and R₁₂ is independently benzyl [F] (sTIP-01) or 2-aminobutyric acid (sTIP-01^{F12&}) or;
wherein R₈ is —(CH₂)₂—C(O)NH₂ [Q] and R₁₂ is —CH₂CH(CH₃)₂ [L] (sTIP-04) or;
wherein R₈ is methyl [A] and R₁₂ is benzyl [F] (sTIP-01^{F8A}).

19. The peptide of any one of claims 1 to 11, wherein the peptide comprises formula III: 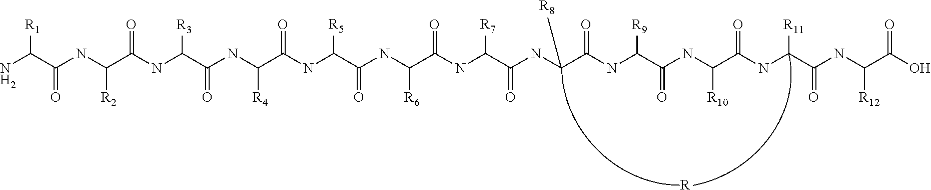
wherein:
R₁ and R₂ are —(CH₂)₄—NH₂ [K];
R₃ is —(CH₂)₃—NH—C(NH₂)₂ [R];
R₄ is —CH₂-Phenyl-OH [Y];
R₅ is —CH₂—OH [S];
R₆ is —(CH₂)₃—NH—C(NH₂)₂ [R];
R₇ is —(CH₂)₂C(O)OH [E], or aminoisobutyric acid;
R₈ and R₁₁ are independently H, a C₁ to C₁₀ alkyl, alkenyl, alkynyl, arylalkyl, cycloalkylalkyl, heteroarylalkyl, or heterocyclylalkyl;
R₉ and R₁₀ are —CH₂CH(CH₃)₂ [L]; and
R₁₂ is benzyl [F], or —CH₂CH(CH₃)₂ [L], or aminoisobutyric acid or 2-aminobutyric acid;
and wherein
R is anyone of alkyl, alkenyl, alkynyl, or [R′—K—R″]n; each of which is substituted with 0-6 R₁₃;
R′ and R″ are independently alkylene, alkenylene or alkynylene;
each R₁₃ is independently halo, alkyl, OR₁₄, N(R₁₄)₂, SR₁₄, SOR₁₄, SO₂R₁₄, CO₂R₁₄, R₁₄, a fluorescent moiety, or a radioisotope;
K is independently O, S, SO, SO₂, CO, CO₂, or CONR₁₄;
each R₁₄ is independently H, alkyl, or a therapeutic agent;
n is an integer from 1-4.

20. The peptide of claim 19, wherein R₈ and R₁₁ are independently H or a C₁ to C₆ alkyl.

21. The peptide of claim 20, wherein R₈ and R₁₁ are each —CH₃ [A] and R is a cyclooctenyl (sTIP-03) cross-linking the α-carbon of the two unnatural amino-acids.

22. The peptide of any one of claims 1 to 11, wherein the peptide comprises formula IV: 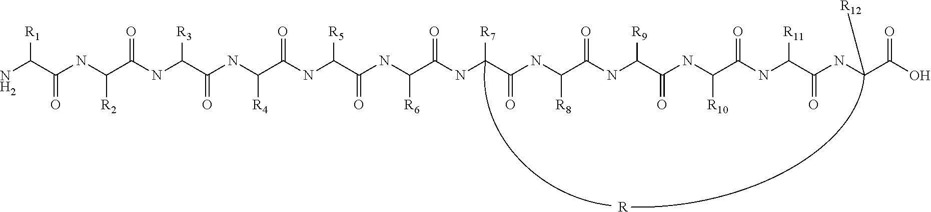
wherein:
R₁ and R₂ are —(CH₂)₄—NH₂ [K];
R₃ is —(CH₂)₃—NH—C(NH₂)₂ [R];
R₄ is —CH₂-Phenyl-OH [Y];
R₅ is —CH₂—OH [S];
R₆ is —(CH₂)₃—NH—C(NH₂)₂ [R];
R₇ and R₁₂ are independently H or a C₁ to C₁₀ alkyl, alkenyl, alkynyl, arylalkyl, cycloalkylalkyl, heteroarylalkyl, or heterocyclylalkyl;
R₈ is benzyl [F], or —(CH₂)₂—C(O)NH₂ [Q], or —CH₃ [A], or aminoisobutyric acid;
R₉ and R₁₀ are —CH₂CH(CH₃)₂ [L]; and
R₁₁ is —H [G] or aminoisobutyric acid;
and wherein
R is anyone of alkyl, alkenyl, alkynyl, or [R′—K—R″]n; each of which is substituted with 0-6 R₁₃;
R′ and R″ are independently alkylene, alkenylene or alkynylene;
each R₁₃ is independently halo, alkyl, OR₁₄, N(R₁₄)₂, SR₁₄, SOR₁₄, SO₂R₁₄, CO₂R₁₄, R₁₄, a fluorescent moiety, or a radioisotope;
K is independently O, S, SO, SO₂, CO, CO₂, or CONR₁₄;
each R₁₄ is independently H, alkyl, or a therapeutic agent;
n is an integer from 1-4.

23. The peptide of claim 22, wherein R₇ and R₁₂ are independently H or a C₁ to C₆ alkyl.

24. The peptide of any one of claims 1 to 11, wherein the peptide comprises formula V: 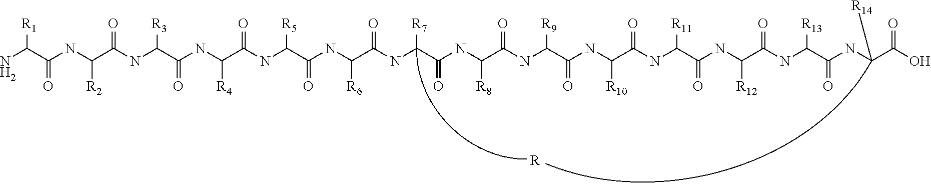
wherein:
R₁ and R₂ are —(CH₂)₄—NH₂ [K];
R₃ is —(CH₂)₃—NH—C(NH₂)2 [R];
R₄ is —CH₂-Phenyl-OH [Y];
R₅ is —CH₂—OH [S];
R₆ is —(CH₂)₃—NH—C(NH₂)₂ [R];
R₇ and R₁₄ are independently H or a C₁ to C₁₀ alkyl, alkenyl, alkynyl, arylalkyl, cycloalkylalkyl, heteroarylalkyl, or heterocyclylalkyl;
R₈ is benzyl [F], or —(CH₂)₂—C(O)NH₂ [Q], or —CH₃ [A], or aminoisobutyric acid;
R₉ and R₁₀ are —CH₂CH(CH₃)₂ [L]; and
R₁₁ is —H [G] or aminoisobutyric acid;
R₁₂ is benzyl [F];
R₁₃ is —(CH₂)₂—C(O)NH₂ [Q];
R₁₄ is benzyl [F];
and wherein
R is anyone of alkyl, alkenyl, alkynyl, or [R′—K—R″]n; each of which is substituted with 0-6 R₁₆;
R′ and R″ are independently alkylene, alkenylene or alkynylene;
each R₁₆ is independently halo, alkyl, OR₁₇, N(R₁₇)₂, SR₁₇, SOR₁₇, SO₂R₁₇, CO₂R₁₇, R₁₇, a fluorescent moiety, or a radioisotope;
K is independently O, S, SO, SO₂, CO, CO₂, or CONR₁₇;
each R₁₇ is independently H, alkyl, or a therapeutic agent;
n is an integer from 1-4.

25. The peptide of claim 24, wherein R₇ and R₁₄ are independently H or a C₁ to C₆ alkyl.

26. The peptide of claim 24, wherein R₇ and R₁₄ are each —CH₃ [A] and R is a 4′-cyclooctenyl (sTIP-05) cross-linking the α-carbon of the two unnatural amino-acids.

27. The peptide of any one of claims 1 to 11, wherein the peptide comprises formula VI: 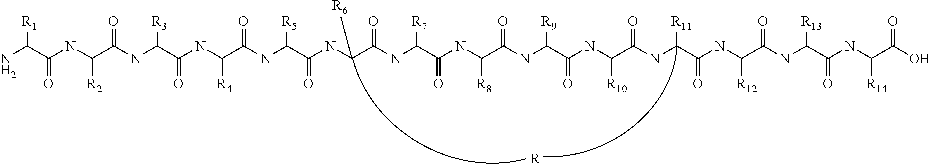
wherein:
R₁ and R₂ are —(CH₂)₄—NH₂ [K];
R₃ is —(CH₂)₃—NH—C(NH₂)2 [R];
R₄ is —CH₂-Phenyl-OH [Y];
R₅ is —CH₂—OH [S];
R₆ and R₁₁ are independently H or a C₁ to C₁₀ alkyl, alkenyl, alkynyl, arylalkyl, cycloalkylalkyl, heteroarylalkyl, or heterocyclylalkyl;
R₇ is —(CH₂)₂C(O)OH [E], or aminoisobutyric acid;
R₈ is benzyl [F], or —(CH₂)₂—C(O)NH₂ [Q], or —CH₃ [A], or aminoisobutyric acid;
R₉ and R₁₀ are —CH₂CH(CH₃)₂ [L];
R₁₂ is benzyl [F];
R₁₃ is —(CH₂)₂—C(O)NH₂ [Q];
R₁₄ is benzyl [F];
and wherein
R is anyone of alkyl, alkenyl, alkynyl, or [R′—K—R″]n; each of which is substituted with 0-6 R₁₆;
R′ and R″ are independently alkylene, alkenylene or alkynylene;
each R₁₆ is independently halo, alkyl, OR₁₇, N(R₁₇)₂, SR₁₇, SOR₁₇, SO₂R₁₇, CO₂R₁₇, R₁₇, a fluorescent moiety, or a radioisotope;
K is independently O, S, SO, SO₂, CO, CO₂, or CONR₁₇;
each R₁₇ is independently H, alkyl, or a therapeutic agent;
n is an integer from 1-4.

28. The peptide of claim 27, wherein R₆ and R₁₁ are independently H or a C₁ to C₆ alkyl.

29. The peptide of claim 27, wherein R₆ and R₁₁ are each —CH₃ [A] and R is a 4′-cyclooctenyl (sTIP-06) cross-linking the α-carbon of the two unnatural amino-acids.

30. The peptide of any one of claims 1 to 11, wherein the peptide comprises formula VII: 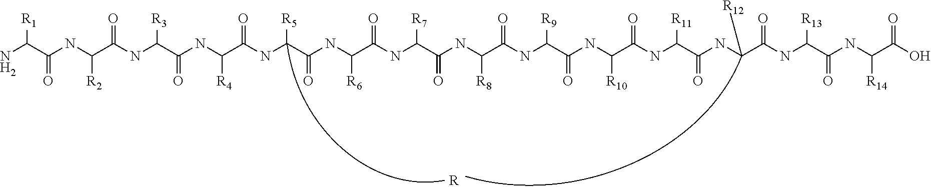
wherein:
R₁ and R₂ are —(CH₂)₄—NH₂ [K];
R₃ is —(CH₂)₃—NH—C(NH₂)2 [R];
R₄ is —CH₂-Phenyl-OH [Y];
R₅ and R₁₂ are independently H or a C₁ to C₁₀ alkyl, alkenyl, alkynyl, arylalkyl, cycloalkylalkyl, heteroarylalkyl, or heterocyclylalkyl;
R₆ is —(CH₂)₃—NH—C(NH₂)₂ [R];
R₇ is —(CH₂)₂C(O)OH [E], or aminoisobutyric acid;
R₈ is benzyl [F], or —(CH₂)₂—C(O)NH₂ [Q], or —CH₃ [A], or aminoisobutyric acid;
R₉ and R₁₀ are —CH₂CH(CH₃)₂ [L];
R₁₁ is —H [G] or aminoisobutyric acid;
R₁₃ is —(CH₂)₂—C(O)NH₂ [Q];
R₁₄ is benzyl [F];
and wherein
R is anyone of alkyl, alkenyl, alkynyl, or [R′—K—R″]n; each of which is substituted with 0-6 R₁₆;
R′ and R″ are independently alkylene, alkenylene or alkynylene;
each R₁₆ is independently halo, alkyl, OR₁₇, N(R₁₇)₂, SR₁₇, SOR₁₇, SO₂R₁₇, CO₂R₁₇, R₁₇, a fluorescent moiety, or a radioisotope;
K is independently O, S, SO, SO₂, CO, CO₂, or CONR₁₇;
each R₁₇ is independently H, alkyl, or a therapeutic agent;
n is an integer from 1-4.

31. The peptide of claim 30, wherein R₅ and R₁₂ are independently H or a C₁ to C₆ alkyl.

32. The peptide of claim 30, wherein R₅ and R₁₂ are each —CH₃ [A] and R is a cyclooctenyl (sTIP-07) cross-linking the α-carbon of the two unnatural amino-acids.

33. The peptide of any one of claims 1 to 11, wherein the peptide comprises formula VIII: 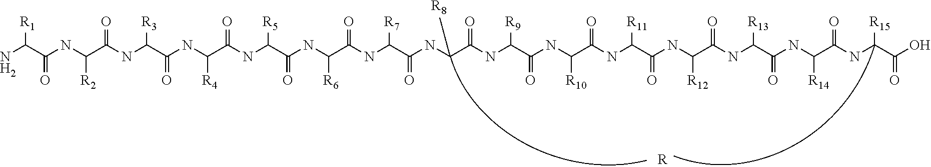
wherein:
R₁ and R₂ are —(CH₂)₄—NH₂ [K];
R₃ is —(CH₂)₃—NH—C(NH₂)2 [R];
R₄ is —CH₂-Phenyl-OH [Y];
R₅ is —CH₂—OH [S];
R₆ is —(CH₂)₃—NH—C(NH₂)₂ [R];
R₇ is —(CH₂)₂C(O)OH [E], or aminoisobutyric acid;
R₈ and R₁₅ are independently H or a C₁ to C₁₀ alkyl, alkenyl, alkynyl, arylalkyl, cycloalkylalkyl, heteroarylalkyl, or heterocyclylalkyl;
R₉ and R₁₀ are —CH₂CH(CH₃)₂ [L];
R₁₁ is —H [G] or aminoisobutyric acid;
R₁₂ is benzyl [F];
R₁₃ is —(CH₂)₂—C(O)NH₂ [Q];
R₁₄ is benzyl [F];
and wherein
R is anyone of alkyl, alkenyl, alkynyl, or [R′—K—R″]n; each of which is substituted with 0-6 R₁₆;
R′ and R″ are independently alkylene, alkenylene or alkynylene;
each R₁₆ is independently halo, alkyl, OR₁₇, N(R₁₇)₂, SR₁₇, SOR₁₇, SO₂R₁₇, CO₂R₁₇, R₁₇, a fluorescent moiety, or a radioisotope;
K is independently O, S, SO, SO₂, CO, CO₂, or CONR₁₇;
each R₁₇ is independently H, alkyl, or a therapeutic agent;
n is an integer from 1-4.

34. The peptide of claim 33, wherein R₈ and R₁₅ are independently H or a C₁ to C₆ alkyl.

35. The peptide of claim 33, wherein R₈ and R₁₅ are each —CH₃ [A] and R is a 4′-cyclooctenyl (sTIP-08) cross-linking the α-carbon of the two unnatural amino-acids.

36. The peptide of any one of claims 1 to 11, selected from the group consisting of:

| | |
|---|---|
| KKRYSRXFLLXF | SEQ ID NO: 2 |
| KKRYSREXLLGX | SEQ ID NO: 3 |
| KKRYSREXLLXF | SEQ ID NO: 4 |
| KKRYSRXQLLXL | SEQ ID NO: 5 |
| KKRYSRXFLLX | SEQ ID NO: 6 |
| KKRYSRXFLLX& | SEQ ID NO: 7 |
| KKRYSRXALLXF | SEQ ID NO: 8 |
| KKRYSR*FLL*F | SEQ ID NO: 9 |
| KKRYSRE*LLG* | SEQ ID NO: 10 |
| KKRYSRE*LL*F | SEQ ID NO: 11 |
| KKRYSR*QLL*L | SEQ ID NO: 12 |
| KKRYSR*FLL* | SEQ ID NO: 13 |
| KKRYSR*FLL*& | SEQ ID NO: 14 |
| KKRYSR*ALL*F | SEQ ID NO: 15 |
| KKRYSR*FLLGFQ* | SEQ ID NO: 17 |
| KKRYS*EFLLGF*F | SEQ ID NO: 18 |
| KKRY*REFLLG*QF | SEQ ID NO: 19 |
| KKRYSRE*LLGFQF* | SEQ ID NO: 20 |

wherein X represents aminoisobutyric acid;
* represent a staple position; and
& represents 2-amino-butyric acid.

37. An isolated nucleic acid molecule encoding a peptide comprising the amino acid sequence KKRYSREFLLGF (SEQ ID NO: 1), wherein the peptide is modified to obtain any one of the peptides referred to in any one of claims 1 to 36.

38. A vector comprising a nucleic acid molecule of claim 37.

39. A host cell comprising a nucleic acid molecule of claim 37 or a vector of claim 38.

40. A pharmaceutical composition comprising a peptide of any one of claims 1 to 36, or an isolated nucleic acid molecule of claim 37, or a vector of claim 38.

41. The pharmaceutical composition of claim 40, further comprising one or more pharmaceutically acceptable excipients, or vehicles, or carriers.

42. The pharmaceutical composition according to claim 40 or 41, wherein the pharmaceutical composition further comprises a therapeutic compound.

43. The pharmaceutical composition of claim 42, wherein the therapeutic compound is an apoptosis promoting compound.

44. The pharmaceutical composition of claim 43, wherein the apoptosis promoting compound is selected from the group consisting of Cyclin-dependent Kinase (CDK) inhibitors, Receptor Tyrosine Kinase (RTK) inhibitors, BCL (B-cell lymphoma) family BH3 (Bcl-2 homology domain 3)-mimetic inhibitors and Ataxia Telangiectasia Mutated (ATM) inhibitors.

45. The pharmaceutical composition of claim 44, wherein the CDK inhibitors comprise inhibitors selected from the group consisting of:
2-(R)-(1-Ethyl-2-hydroxyethylamino)-6-benzylamino-9-isopropylpurine (CYC202; Roscovitine; Seliciclib);
4-[[5-Amino-1-(2,6-difluorobenzoyl)-1H-1,2,4-triazol-3-yl]amino]benzenesulfonamide (JNJ-7706621);
N-(4-piperidinyl)-4-(2,6-dichlorobenzoylamino)-1H-pyrazole-3-carboxamide (AT-7519);
N-(5-(((5-(1,1-dimethylethyl)-2-oxazolyl)methyl)thio)-2-thiazolyl)-4-piperidinecarboxamide (SNS-032);
8,12-Epoxy-1H,8H-2,7b,12a-triazadibenzo(a,g)cyclonona(cde)triinden-1-one, 2,3,9,10,11,12-hexahydro-3-hydroxy-9-methoxy-8-methyl-10-(methylamino)-(UCN-01; 7-Hydroxystaurosporine; KRX-0601);
N,1,4,4-tetramethyl-8-((4-(4-methylpiperazin-1-yl)phenyl)amino)-4,5-dihydro-1H-pyrazolo[4,3-h]quinazoline-3-carboxamide (PHA-848125; milciclib);
2-(2-chlorophenyl)-5,7-dihydroxy-8-[(3S,4R)-3-hydroxy-1-methylpiperidin-4-yl]chromen-4-one hydrochloride (flavopiridol; alvocidib);
6-acetyl-8-cyclopentyl-5-methyl-2-((5-(piperazin-1-yl)pyridin-2-yl)amino)pyrido[2,3-d]pyrimidin-7(8H)-one hydrochloride (PD 0332991);
4-(1-isopropyl-2-methyl-1H-imidazol-5-yl)-N-(4-(methylsulfonyl)phenyl)pyrimidin-2-amine (AZD5438);
(S)-3-(((3-ethyl-5-(2-(2-hydroxyethyl)piperidin-1-yl)pyrazolo[1,5-a]pyrimidin-7-yl)amino)methyl)pyridine 1-oxide (Dinaciclib; SCH 727965);
N-(4-Piperidinyl)-4-(2,6-dichlorobenzoylamino)-1H-pyrazole-3-carboxamide hydrochloride (AT-7519); and
pharmaceutically acceptable salts thereof.

46. The pharmaceutical composition of claim 44, wherein the RTK inhibitors comprise inhibitors selected from the group consisting of:
N-[3-chloro-4-[(3-fluorophenyl)methoxy]phenyl]-6-[5-[(2-methylsulfonylethylamino)methyl]-2-furyl]quinazolin-4-amine (lapatinib);
N1′-[3-fluoro-4-[[6-methoxy-7-(3-morpholinopropoxy)-4-quinolyl]oxy]phenyl]-N1-(4-fluorophenyl)cyclopropane-1,1-dicarboxamide (foretinib);
N-(4-((6,7-Dimethoxyquinolin-4-yl)oxy)phenyl)-N-(4-fluorophenyl)cyclopropane-1,1-dicarboxamide (cabozantinib(XL184));
N-(4-((6,7-Dimethoxyquinolin-4-yl)oxy)phenyl)-N-(4-fluorophenyl)cyclopropane-1,1-dicarboxamide (cabozantinib(XL184));
3-[(1R)-1-(2,6-dichloro-3-fluorophenyl)ethoxy]-5-(1-piperidin-4-ylpyrazol-4-yl)pyridin-2-amine (crizotinib (Xalkori));
(3Z)—N-(3-Chlorophenyl)-3-({3,5-dimethyl-4-[(4-methylpiperazin-1-yl)carbonyl]-1H-pyrrol-2-yl}methylene)-N-methyl-2-oxo-2,3-dihydro-1H-indole-5-sulfonamide (SU11274);
(3Z)-5-[[(2,6-Dichlorophenyl)methyl]sulfonyl]-3-[[3,5-dimethyl-4-[[(2R)-2-(1-pyrrolidinylmethyl)-1-pyrrolidinyl]carbonyl]-1H-pyrrol-2-yl]methylene]-1,3-dihydro-2H-indol-2-one hydrate (PHA-665752);
6-[[6-(1-Methylpyrazol-4-yl)-[1,2,4]triazolo[4,3-b]pyridazin-3-yl]sulfanyl]quinoline (SGX-523);
4-[1-(6-Quinolinylmethyl)-1H-1,2,3-triazolo[4,5-b]pyrazin-6-yl]-1H-pyrazole-1-ethanol methanesulfonate (1:1) (PF-04217903);
2-Fluoro-N-methyl-4-[7-[(quinolin-6-yl)methyl]imidazo[1,2-b]-[1,2,4]triazin-2-yl]benzamide (INCB28060);
N-[4-[(3-Chloro-4-fluorophenyl)amino]-7-[[(3S)-tetrahydro-3-furanyl]oxy]-6-quinazolinyl]4(dimethylamino)-2-butenamide (afatinib);
3-(5,6-Dihydro-4H-pyrrolo[3,2,1-ij]quinolin-1-yl)-4-(1H-indol-3-yl)-pyrrolidine-2,5-dione (ARQ-197 (Tivantinib));
N-[(2R)-1,4-dioxan-2-ylmethyl]-N-methyl-N′-[3-(1-methyl-1H-pyrazol-4-yl)-5-oxo-5H-benzo[4,5]cyclohepta[1,2-b]pyridin-7-yl]sulfuric diamide (MK-2461);
N-[4-(3-Amino-1H-indazol-4-yl)phenyl]-N′-(2-fluoro-5-methylphenyl)urea (Linifanib(ABT 869));
4-[[(3S)-3-Dimethylaminopyrrolidin-1-yl]methyl]-N-[4-methyl-3-[(4-pyrimidin-5-ylpyrimidin-2-yl)amino]phenyl]-3-(trifluoromethyl)benzamide (Bafetinib (INNO-406)); and
pharmaceutical acceptable salts thereof.

47. The pharmaceutical composition of claim 44, wherein the BCL family BH3-mimetic inhibitors comprise inhibitors selected from the group consisting of:
4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (ABT 263; Navitoclax);
2-[2-[(3,5-Dimethyl-1H-pyrrol-2-yl)methylene]-3-methoxy-2H-pyrrol-5-yl]-1H-indole methanesulfonate (Obatoclax mesylate (GX15-070));
4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-Benzamide (ABT-737); and
pharmaceutically acceptable salts thereof.

48. The pharmaceutical composition of claim 44, wherein the ATM inhibitors comprise inhibitors selected from the group consisting of:
2-Morpholin-4-yl-6-thianthren-1-yl-pyran-4-one (KU-55933);
(2R,6S)-2,6-Dimethyl-N-[5-[6-(4-morpholinyl)-4-oxo-4H-pyran-2-yl]-9H-thioxanthen-2-yl]-4-morpholineacetamide (KU-60019);
1-(6,7-Dimethoxy-4-quinazolinyl)-3-(2-pyridinyl)-1H-1,2,4-triazol-5-amine (CP466722);
α-Phenyl-N-[2,2,2-trichloro-1-[[[(4-fluoro-3-nitrophenyl)amino]thioxomethyl]amino]ethyl]benzene acetamide (CGK 733)
and pharmaceutically acceptable salts thereof.

49. Use of the peptide according to any one of claims 1 to 36 in the manufacture of a medicament for treating or preventing cancer.

50. The use of claim 49, wherein the cancer is characterized by overexpression or hyperactivity of eIF4E containing complexes.

51. The use according to claim 49, wherein cancer is selected from a group comprising or consisting of gastric cancer, colon cancer, lung cancer, breast cancer, bladder cancer, neuroblastoma, melanoma, head and neck cancer, esophagus cancer, cervix cancer, prostate cancer and leukemia.

52. Method of treating or preventing cancer in a patient comprising administering a pharmaceutically effective amount of the peptide of any one of claims 1 to 36 or the isolated nucleic acid molecule of claim 37, or the vector according to claim 38.

53. The method according to claim 52 wherein the method comprises the administration of one or more further therapeutic agents to the patient, wherein administration is simultaneous, sequential or separate.

54. Use of a peptide according to any one of claims 1 to 36 for protein purification, or for inhibiting protein-protein interactions, or as template for protein-protein interactions.
